(19) United States
(12) Patent Application Publication
(10) Pub. No.: US 2003/0166008 A1
(43) Pub. Date: Sep. 4, 2003

(54) Nucleic acid encoding a G-protein-coupled receptor, and uses thereof

(76) Inventors: Haifeng Eishingdrelo, Montville, NJ (US); Holly Dressler, Franklin Park, NJ (US); Jidong Cai, Whippany, NJ (US); Paul Wright, New Hope, PA (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/366,504

(22) Filed: Feb. 13, 2003

Related U.S. Application Data
(60) Provisional application No. 60/356,686, filed on Feb. 14, 2002.

(30) Foreign Application Priority Data
Aug. 22, 2002 (GB) 0219574.1

(51) Int. Cl. G01N 33/53; C07H 21/04; C12P 21/02; C12N 5/06; C07K 14/705

(52) U.S. Cl. 435/7.1; 435/69.1; 435/320.1; 435/325; 530/350; 530/388.22; 536/23.5

(57) ABSTRACT

Provided herein is a novel and useful G-protein coupled receptor that is involved in signal transduction with respect to inflammation and physiological immunological response. Also provided are methods of using the receptor to screen for molecules that may modulate the activity of the receptor. Such molecules may readily have applications in treating a plethora of inflammation and immunologically related diseases and disorders.

### PRIORITY CLAIM

This application claims priority under 35 U.S.C. 119 from U.S. S. No. 60/356,686 filed on Feb. 14, 2002 and British Application No. 0219574.1 filed on Aug. 22, 2002, wherein said applications are hereby incorporated by reference herein in their entireties.

### FIELD OF THE INVENTION

The present invention relates generally to a novel nucleic acid molecule that encodes for GAVE19, a heretofore unknown G-protein-coupled receptor, along with uses of the nucleic acid molecule and GAVE19.

### BACKGROUND OF THE INVENTION

The G protein-coupled receptors (GPCRs) are a large family of integral membrane proteins that are involved in cellular signal transduction. GPCRs respond to a variety of extracellular signals, including neurotransmitters, hormones, odorants and light, and are capable of transducing signals so as to initiate a second messenger response within the cell. Many therapeutic drugs target GPCRs because those receptors mediate a wide variety of physiological responses, including inflammation, vasodilation, heart rate, bronchodilation, endocrine secretion and peristalsis.

GPCRs are characterized by extracellular domains, seven transmembrane domains and intracellular domains. Some of the functions the receptors perform, such as binding ligands and interacting with G proteins, are related to the presence of certain amino acids in critical positions. For example, a variety of studies have shown that differences in amino acid sequence in GPCRs account for differences in affinity to either a natural ligand or a small molecule agonist or antagonist. In other words, minor differences in sequence can account for different binding affinities and activities. (See, for example, Meng et al., J Bio Chem (1996) 271(50):32016-20; Burd et al., J Bio Chem (1998) 273(51):34488-95; and Hurley et al., J Neurochem (1999) 72(1):413-21). In particular, studies have shown that amino acid sequence differences in the third intracellular domain can result in different activities. Myburgh et al. found that alanine 261 of intracellular loop 3 of gonadotropin releasing hormone receptor is crucial for G protein coupling and receptor internalization (Biochem J (1998) 331(Part 3):893-6). Wonerow et al. studied the thyrotropin receptor and demonstrated that deletions in the third intracellular loop resulted in constitutive receptor activity (J Bio Chem (1998)273(14):7900-5).

In general, the action of the binding of an endogenous ligand to a receptor results in a change in the conformation of the intracellular domain(s) of the receptor allowing for coupling between the intracellular domain(s) and an intracellular component, a G-protein. Several G proteins exist, such as G_{q}, Gₛ, Gᵢ, G_{z} and Gₒ (see, e.g. Dessauer et al., Clin Sci (Colch) (1996) 91(5):527-37). The IC-3 loop as well as the carboxy terminus of the receptor interact with the G proteins (Pauwels et al., Mol Neurobiol (1998) 17(1-3):109-135 and Wonerow et. al., supra). Some GPCRs are “promiscuous” with respect to G proteins, i.e., a GPCR can interact with more than one G protein (see, e.g., Kenakin, Life Sciences (1988) 43:1095).

Ligand activated GPCR coupling with G protein begins a signaling cascade process (referred to as “signal transduction”). Such signal transduction ultimately results in cellular activation or cellular inhibition.

GPCRs exist in the cell membrane in equilibrium between two different conformations: an “inactive” and an “active” state. A receptor in an inactive state is unable to link to the intracellular signaling transduction pathway to produce a biological response (exceptions exist, such as during over-expression of receptor in transduced cells, see e.g., www.creighton.edu/Pharmacology/inverse.htm.). Modulation of the conformation to the active state allows linkage to the transduction pathway (via the G protein) and produces a biological response. Agonists bind and make the active conformation much more likely. However, sometimes, if there is already a considerable response in the absence of any agonist, such receptors are said to be constitutively active (i.e., already in an active conformation or ligand independent or autonomous active state). When agonists are added to such systems, an enhanced response routinely is observed. However, when a classical antagonist is added, binding by such molecules produces no effect. On the other hand, some antagonists cause an inhibition of the constitutive activity of the receptor, suggesting that the latter class of drugs technically are not antagonists but are agonists with negative intrinsic activity. Those drugs are called inverse agonists, www.creighton.edu/Pharmacology/inverse.htm.).

Traditional study of receptors has proceeded from the assumption that the endogenous ligand first be identified before discovery could move forward to identify antagonists and other receptor effector molecules. Even where antagonists might have been discovered first, the dogmatic response was to identify the endogenous ligand (WO 00/22131). However, as the active state is the most useful for assay screening purposes, obtaining such constitutive receptors, especially GPCRs, would allow for the facile isolation of agonists, partial, agonists, inverse agonists and antagonists in the absence of information concerning endogenous ligands. Moreover, in diseases that result from disorders of receptor activity, drugs that cause inhibition of constitutive activity, or more specifically, reduce the effective activated receptor concentration, could be discovered more readily by assays using receptors in the autonomous active state. For example, as receptors that may be transfected into patients to treat disease, the activity of such receptors may be fine-tuned with inverse agonists discovered by such assays.

Diseases such as asthma, chronic obstructive pulmonary disease (COPD) and rheumatoid arthritis (RA) generally are considered to have an inflammatory etiology involving T helper cells, monocyte-macrophages and eosinophils. Current anti-inflammatory therapy with corticosteroids is effective in asthma but is associated with metabolic and endocrine side effects. The same is possibly true for inhaled formulations that can be absorbed through lung or nasal mucosa. Satisfactory oral therapies for RA or COPD currently are lacking.

Eosinophils mediate much of the airway dysfunction in allergy and asthma. Interleukin-5 (IL-5) is an eosinophil growth and activating cytokine. Studies have shown IL-5 to be necessary for tissue eosinophilia and for eosinophil-mediated tissue damage resulting in airway hyperresponsiveness (Chang et al., J Allergy Clin Immunol (1996) 98(5 pt 1):922-931 and Duez et al., Am J Respir Crit Care Med (2000) 161(1):200-206). IL-5 is made by T-helper-2 cells (Th2) following allergen (e.g. house dust mite antigen) exposure in atopic asthma.

RA is believed to result from accumulation of activated macrophages in the affected synovium. Interferon γ (IFNγ) is a T-helper-1 (Th1) cell-derived cytokine with numerous proinflammatory properties. It is the most potent macrophage activating cytokine and induces MHC class II gene transcription contributing to a dendritic cell-like phenotype.

Lipopolysaccharide (LPS) is a component of gram-negative bacterial cell walls that elicits inflammatory responses, including tumor necrosis factor α (TNFα) release. The efficacy of intravenous anti-TNFα therapy in RA has been demonstrated in the clinic. COPD is thought also to result from macrophage accumulation in the lung, the macrophages produce neutrophil chemoattractants (e.g., IL-8: de Boer et al., J Pathol (2000) 190(5):619-626). Both macrophages and neutrophils release cathepsins that cause degradation of the alveolar wall. It is believed that lung epithelium can be an important source for inflammatory cell chemoattractants and other inflammatory cell-activating agents (see, for example, Thomas et al., J Virol (2000) 74(18):8425-8433; Lamkhioued et al., Am J Respir Crit Care Med (2000) 162(2 Pt. 1):723-732; and Sekiya et al., J Immunol (2000) 165(4):2205-2213).

Given the role GPCRs have in disease and the ability to treat diseases by modulating the activity of GPCRs, identification and characterization of previously unknown GPCRs can provide for the development of new compositions and methods for treating disease states that involve the activity of a GPCR. Accordingly, what is needed is the discovery, isolation and characterization of novel and useful nucleic acid molecules that encode for heretofore unknown GPCRs.

What is also needed are assays that utilize such heretofore unknown GPCRs to identify molecules that can serve potential agonists or antagonists of GPCRS. These molecules may readily have applications as therapeutic agents for modulating the activity of GPCRs in vivo, and thus, treat a plethora of diseases related to GPCR activity.

The citation of any reference herein should not be construed as an admission that such reference is available as “Prior Art” to the instant application.

### SUMMARY OF THE INVENTION

The instant invention identifies and characterizes the expression of a novel constitutively active murine GPCR, GAVE19, and provides compositions and methods for applying the discovery to the identification and treatment of related diseases.

Thus broadly, the present invention extends to an isolated nucleic acid molecule comprising a DNA sequence of FIG. 1 (SEQ ID NO:1), a variant thereof, a fragment thereof, or an analog or a derivative thereof. Such a variant of the present invention may be an allelic variant, a degenerate variant, or an allelic variant that results in a degenerate change in the sequence.

Moreover, the present invention extends to an isolated nucleic acid molecule hybridizable to the isolated nucleic acid molecule of SEQ ID NO:1, or a variant thereof, under stringent hybridization conditions. Yet further, the present invention extends to an isolated nucleic acid molecule hybridizable to a nucleic acid molecule that is complementary to the DNA sequence of SEQ ID NO:1 under stringent hybridization conditions. Stringent hybridization conditions are described infra.

Furthermore, the present invention extends to an isolated nucleic acid molecule comprising a DNA sequence that encodes a polypeptide comprising an amino acid sequence of SEQ ID NO:2.

Optionally, an isolated nucleic acid molecule of the present invention as described above may be detectably labeled. Examples of detectable labels having applications herein include, but certainly are not limited to an enzyme, a radioactive isotope, or a chemical which fluoresces. Particular examples of detectable labels are described infra.

Particular polypeptides are also encompassed within the present invention. For example, the present invention extends to a purified polypeptide comprising the amino acid sequence of SEQ ID NO:2, a conservative variant thereof, or an analog or derivative thereof. Optionally, a polypeptide of the present invention may be detectably labeled.

In addition, the present invention extends to antibodies wherein a polypeptide of the present invention is the immunogen used in production of the antibodies. These antibodies can be monoclonal or polyclonal. Moreover, the antibodies can be “chimeric” as, for example, they may comprise protein domains of antibodies raised against a purified polypeptide of the present invention in different species. Naturally, an antibody of the present invention may be detectably labeled. Particular examples of detectable labels having applications herein are described infra.

The present invention further extends to an expression vector comprising a nucleic acid molecule comprising a DNA sequence of SEQ ID NO:1, a variant thereof, an analog or derivative thereof, or a fragment thereof, operatively associated with an expression control element. Furthermore, an expression vector of the present invention may comprise an isolated nucleic acid molecule hybridizable under stringent hybridization conditions to an isolated nucleic acid molecule comprising a DNA sequence of SEQ ID NO:1, operatively associated with an expression control element, or is hybridizable under stringent hybridization conditions to a hybridization probe that is complementary to an isolated nucleic acid molecule comprising a DNA sequence of SEQ ID NO:1, wherein the hybridization probe is operatively associated with an expression control element. A particular example of an expression control element having applications herein is a promoter. Examples of particular promoters applicable to the present invention, include, but are not limited to early promoters of hCMV, early promoters of SV40, early promoters of adenovirus, early promoters of vaccinia, early promoters of polyoma, late promoters of SV40, late promoters of adenovirus, late promoters of vaccinia, late promoters of polyoma, the lac system, the trp system, the TAC system, the TRC system, the major operator and promoter regions of phage lambda, control regions of fd coat protein, 3-phosphoglycerate kinase promoter, acid phosphatase promoter, or promoters of yeast α mating factor, to name only a few.

With an expression vector of the present invention, one may transfect or transform a host cell and produce a polypeptide comprising an amino acid sequence of SEQ ID NO:2, or a variant thereof. The host cell may be either a prokaryotic cell or a eukaryotic cell. Particular examples of unicellular hosts having applications herein include E. coli, Pseudonomas, Bacillus, Strepomyces, yeast, CHO, R1.1, B-W, L-M, COS1, COS7, BSC1, BSC40, BMT10 and Sf9 cells, etc.

Moreover, the present invention further extends to a method for producing a purified polypeptide comprising the amino acid sequence of SEQ ID NO:2, a variant thereof, or a fragment thereof. Such a method comprises culturing a host cell transformed or transfected with an expression vector of the present invention under conditions that provide for expression of the purified polypeptide, and then recovering the purified polypeptide from the unicellular host, the culture surrounding the host cell, or from both.

The present invention also extends to assays for identifying compounds that can modulate the activity of GAVE19. Such compounds can be an agonist, an antagonist, or an inverse agonist of GAVE19. Hence accordingly, the present invention extends to a method for identifying an agonist of GAVE19 comprising contacting a potential agonist with a cell expressing GAVE19 in the presence of an endogenous ligand, and determining whether the signaling activity of GAVE19 is increased when the potential agonist is present, relative to the signaling activity of GAVE19 in the absence of the potential agonist. Likewise, the present invention extends to a method for identifying an inverse agonist of GAVE19. Such a method comprises contacting a potential inverse agonist with a cell expressing GAVE19, and determining whether the signaling activity of GAVE19 in the presence of the potential inverse agonist and an endogenous ligand or agonist is decreased relative to the signaling activity of GAVE19 under conditions in which the presence of an endogenous ligand or agonist, but in absence of potential inverse agonist, and is decreased in the presence of an endogenous ligand or agonist.

Naturally, the present invention extends to methods for identifying an antagonist of GAVE19. Such a method comprises the steps of contacting a potential antagonist with a cell expressing GAVE19, and determining whether in the presence of said potential antagonist the signaling activity of GAVE 19 is decreased relative to the activity of GAVE 19 in the presence of an endogenous ligand or agonist.

Accordingly, it is an aspect of the present invention to provide an isolated nucleic acid sequence which encodes a GAVE19 protein, a fragment thereof, or a variant thereof.

It is also an aspect of the present invention to provide a variant of an nucleic acid molecule comprising a DNA sequence of SEQ ID NO:1, as well as a DNA molecule that is hybridizable to SEQ ID NO:1 under stringent conditions.

It is a further aspect of the present invention to provide an amino acid sequence for GAVE19, along with variant thereof, a fragment thereof, or an analog or derivative thereof. It is a further aspect of the present invention to provide an expression vector comprising a DNA sequence that encodes GAVE19, a variant thereof, a fragment thereof, or an analog or derivative thereof, wherein the DNA sequence is operably associated with an expression control element.

It is still a further aspect of the present invention to provide an antibody having GAVE19, an variant thereof, an analog or derivative thereof, or a fragment thereof, as an immunogen. Yet another aspect of the present invention involves methods for identify compounds that can modulate the activity of GAVE19 protein. Such modulators may be an antagonist of GAVE19, an agonist of GAVE19, or inverse agonist of GAVE19. Moreover, compounds that modulate the expression or activity of GAVE 19 in mice may well have applications in treating a plethora of diseases or disorders such as various inflammatory diseases, asthma, chronic obstructive pulmonary disease (COPD), and rheumatoid arthritis, to name only a few.

These and other aspects of the present invention will be better appreciated by reference to the following drawings and Detailed Description.

### BRIEF DESCRIPTION OF THE DRAWINGS

FIG. 1: DNA sequence that encodes GAVE 19 (SEQ ID NO:1).

FIG. 2: Amino acid sequence of GAVE19 (SEQ ID NO:2)

FIG. 3: GAVE19 Expression Profile on MPD1.1.2

FIG. 4: Comparison of the amino acid sequence of GAVE19 (SEQ ID NO:2) with the human ortholog GAVE18 (SEQ ID NO 7).

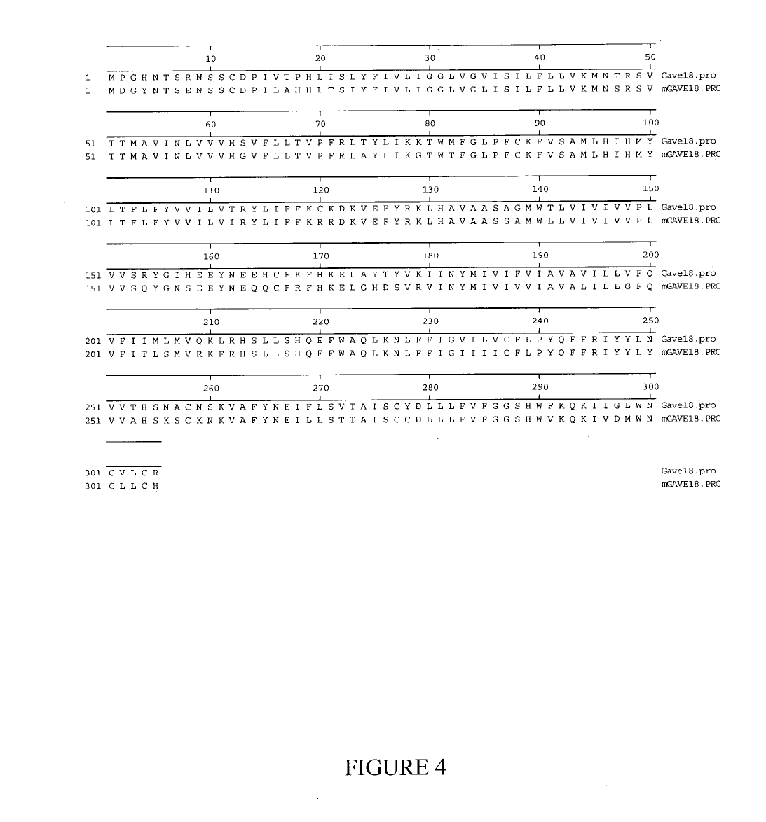

### DETAILED DESCRIPTION OF THE INVENTION

As explained above, the present invention relates to the surprising and unexpected discovery of a heretofore unknown murine nucleic acid molecule that encodes a heretofore unknown G protein-coupled receptor referred to herein as GAVE19. In particular, it has been discovered that GAVE19 is expressed in immune tissues or organs, such as the kidney, liver and small intestine. Hence, GAVE19 can readily serve as a target for the development of pharmaceutical compositions to treat a variety inflammation diseases, such as asthma, rheumatoid arthritis, COPD, etc.

Various terms and phrases used throughout the instant Specification and claims to describe the present invention are set forth below:

As used herein, the term “modulator” refers to a moiety (e.g., but not limited to a ligand and a candidate compound) that modulates the activity of GAVE19. A modulator of the present invention may be an agonist, a partial agonist, an antagonist, or an inverse agonist of GAVE19.

As used herein, the term “agonist” refers to moieties (e.g., but not limited to ligands and candidate compounds) that activate the intracellular response when bound to the receptor, or enhance GTP binding to membranes.

As used herein, the term “partial agonist” refers to moieties (e.g., but not limited to ligands and candidate compounds) that activate the intracellular response when bound to the receptor to a lesser degree/extent than do agonists, or enhance GTP binding to membranes to a lesser degree/extent than do agonists.

As used herein, the term “antagonist” refers moieties (e.g., but not limited to ligands and candidate compounds) that competitively bind to the receptor at the same site as does an agonist. However, an antagonist does not activate the intracellular response initiated by the active form of the receptor and thereby can inhibit the intracellular responses by agonists or partial agonists. In a related aspect, antagonists do not diminish the baseline intracellular response in the absence of an agonist or partial agonist.

As used herein, the term “inverse agonist” refers to moieties (e.g., but not limited to ligand and candidate compound) that bind to a constitutively active receptor and inhibit the baseline intracellular response. The baseline response is initiated by the active form of the receptor below the normal base level of activity that is observed in the absence of agonists or partial agonists, or decrease of GTP binding to membranes.

As used herein, the term “candidate compound” refers to a moiety (e.g., but not limited to a chemical compound) that is amenable to a screening technique. In one embodiment, the term does not include compounds that were publicly known to be compounds selected from the group consisting of agonist, partial agonist, inverse agonist or antagonist of GAVE19. Those compounds were identified by traditional drug discovery processes involving identification of an endogenous ligand specific for a receptor, and/or screening of candidate compounds against a receptor wherein such a screening requires a competitive assay to assess efficacy.

As used herein, the terms “constitutively activated receptor” or “autonomously active receptor,” are used herein interchangeably, and refer to a receptor subject to activation in the absence of ligand. Such constitutively active receptors can be endogenous (e.g., GAVE19) or non-endogenous; i.e., GPCRs that can be modified by recombinant means to produce mutant constitutive forms of wild-type GPCRs (e.g., see EP 1071701; WO 00/22129; WO 00/22131; and U.S. Pat. Nos. 6,150,393 and 6,140,509 which are hereby incorporated by reference herein in their entireties.

As used herein, the term “constitutive receptor activation” refers to the stabilization of a receptor in the active state by means other than binding of the receptor with the endogenous ligand or chemical equivalent thereof.

As used herein, the term “ligand” refers to a moiety that binds to another molecule, wherein the moiety includes, but certainly is not limited to a hormone or a neurotransmitter, and further, wherein the moiety stereoselectively binds to a receptor.

As used herein, the term “family,” when referring to a protein or a nucleic acid molecule of the invention, is intended to mean two or more proteins or nucleic acid molecules having a seemingly common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that second protein. Members of a family also may have common functional characteristics.

As used herein interchangeably, the terms “GAVE19 activity”, “biological activity of GAVE19” and “functional activity of GAVE19”, refer to an activity exerted by a GAVE19 protein, polypeptide or nucleic acid molecule on a GAVE19 responsive cell as determined in vivo or in vitro, according to standard techniques. A GAVE19 activity can be a direct activity, such as an association with or an enzymatic activity on a second protein or an indirect activity, such as a cellular signaling activity mediated by interaction of the GAVE19 protein with a second protein. In a particular embodiment, a GAVE19 activity includes, but is not limited to at least one or more of the following activities: (i) the ability to interact with proteins in the GAVE19 signaling pathway; (ii) the ability to interact with a GAVE19 ligand; and (iii) the ability to interact with an intracellular target protein.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage or cosmid, to name only a few, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control. Particular examples of vectors are described infra.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Preferably, the heterologous DNA includes a gene foreign to the cell.

“Homologous recombination” refers to the insertion of a foreign DNA sequence of a vector into a chromosome. In particular, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

### Isolated Nucleic Acid Molecules of the Present Invention

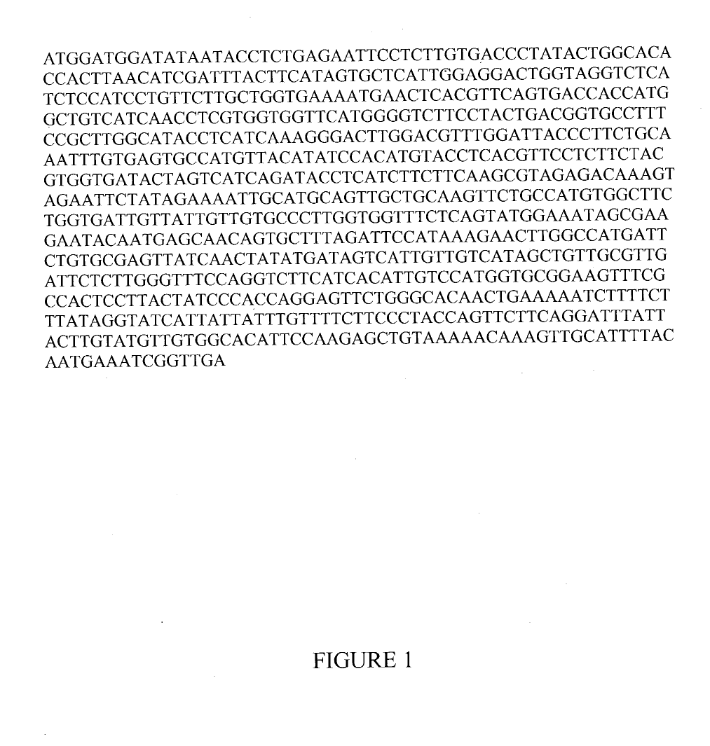

In one aspect, the present invention extends to an isolated nucleic acid molecule comprising DNA sequence of FIG. 1 (SEQ ID NO:1), a variant thereof, a fragment thereof, or an analog or derivative thereof.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

An “isolated” nucleic acid molecule is one that is separated from other nucleic acid molecules present in the natural source of the nucleic acid. In particular, an “isolated” nucleic acid is free of sequences that naturally flank the nucleic acid encoding GAVE19 (i.e., sequences located at the 5′ and 3′ ends of the nucleic acid) in the genomic DNA of the organism from which the nucleic acid is derived. In various embodiments, the isolated GAVE19 nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques or substantially free of chemical precursors or other chemicals when synthesized chemically.

A nucleic acid molecule of the present invention, e.g., a nucleic acid molecule having the nucleotide sequence of SEQ ID NO:1 or a fragment or complement of any of that nucleotide sequence, or an analog or derivative thereof, can be isolated using standard molecular biology techniques and the sequence information provided herein. Using all or a portion of the nucleic acid sequence of SEQ ID NO:1 as a hybridization probe, GAVE19 nucleic acid molecules can be isolated using standard hybridization and cloning techniques (e.g., as described in Sambrook et al).

A nucleic acid molecule of the invention can be amplified using cDNA, mRNA or genomic DNA as a template and appropriate oligonucleotide primers according to standard PCR amplification techniques. Such primers may be readily made using information set forth in SEQ ID NO:1, and routine laboratory techniques. The nucleic acid so amplified can be cloned into an appropriate vector and characterized by DNA sequence analysis. Furthermore, oligonucleotides corresponding to GAVE19 nucleotide sequences can be prepared by standard synthetic techniques, e.g., using an automated DNA synthesizer.

### Isolated nucleic Acid molecule hybridizable to GAVE19 DNA

The present invention further extends to isolated nucleic acid molecules hybridizable to GAVE19 DNA, hybridizable to a hybridization probe that is complementary under stringent hybridization conditions to GAVE19 DNA, or hybridizable under stringent hybridization conditions to both. In particular, the present invention extends to an isolated nucleic acid molecule that is hybridizable under stringent hybridization conditions to a nucleic acid molecule comprising a DNA sequence of SEQ ID NO:1, or to a probe that is complementary to an isolated nucleic acid molecule comprising a DNA sequence of SEQ ID NO:1.

A nucleic acid molecule is “hybridizable” to another nucleic acid molecule, such as a cDNA, genomic DNA, or RNA, when a single stranded form of the nucleic acid molecule can anneal to another nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid molecule is at least about 20 nucleotides; particularly at least about 30 nucleotides; more particularly at least about 40 nucleotides, even more particularly about 50 nucleotides, and yet more particularly at least about 60 nucleotides. In a particular embodiment of the present invention, a hybridizable nucleic acid molecule of the invention is at least 300, 325, 350, 375, 400, 425, 450, 500, 550, 600, 650, 700, 800, 900, 1000 or 1100 nucleotides in length and hybridizes under stringent conditions to the nucleic acid molecule comprising the nucleotide sequence, preferably the coding sequence, of SEQ ID NO:1 a complement thereof, or a fragment thereof.

As used herein, the term “hybridizes under stringent conditions” is intended to describe conditions for hybridization and washing under which nucleotide sequences at least 55%, 60%, 65%, 70% and preferably 75% or more complementary to each other typically remain hybridized. Such stringent conditions are known to those skilled in the art and can be found in “Current Protocols in Molecular Biology”, John Wiley & Sons, N.Y. (1989), 6.3.1-6.3.6. A preferred, non-limiting example of stringent hybridization conditions are hybridization in 6×sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1 or the complement thereof corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein). The skilled artisan will appreciate that the conditions may be modified in view of sequence-specific variables (e.g., length, G-C richness etc.).

The invention contemplates encompassing nucleic acid fragments of GAVE19 that are diagnostic of GAVE19-like molecules that have similar properties. The diagnostic fragments can arise from any portion of the GAVE19 gene including flanking sequences. The fragments can be used as probe of a library practicing known methods.

Moreover, a nucleic acid molecule of the invention can comprise only a portion of a nucleic acid sequence encoding GAVE19, for example, a fragment that can be used as a probe or primer, or a fragment encoding a biologically active portion of GAVE19. For example, such a fragment can comprise, but is not limited to, a region encoding amino acid residues about 1 to about 14 of SEQ ID NO:2. The nucleotide sequence determined from the cloning of the human GAVE19 gene allows for the generation of probes and primers for identifying and/or cloning GAVE19 homologues in other cell types, e.g., from other tissues, as well as GAVE19 homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SEQ ID NO:1 or of a naturally occurring mutant of SEQ ID NO:1. Probes based on a GAVE19 nucleotide sequence can be used to detect transcripts or genomic sequences encoding the similar or identical proteins.

As used herein, the terms “fragment” or “portion” of an isolated nucleic acid molecule of the present invention comprise at least 12, particularly about 25, more particularly about 50, 75, 100, 125, 150, 175, 200, 250, 300, 350 or 400 consecutive nucleotides. Consequently, a “fragment” of an isolated nucleic acid molecule of the present invention is not merely 1 or 2 nucleotides.

Similarly, a “fragment” or “portion” of a polypeptide of the present invention comprises at least 9 contiguous amino acid residues. A particular example of a fragment of a polypeptide of the present invention comprises an epitope to which a GAVE19 antibody, or fragment thereof, binds.

A nucleic acid fragment encoding a “biologically active portion of GAVE19” can be prepared by isolating a portion of SEQ ID NO:1 that encodes a polypeptide having a GAVE19 biological activity, expressing the encoded portion of GAVE19 protein (e.g., by recombinant expression in vitro) and assessing the activity of the encoded portion of GAVE19. The invention further encompasses nucleic acid molecules that differ from the nucleotide sequence of SEQ ID NO:1 due to degeneracy of the genetic code, and thus encode the same GAVE19 protein as that encoded by the nucleotide sequence shown in SEQ ID NO:1.

### Homologous Nucleic Acid Molecules

The present invention further extends to an isolated nucleic acid molecule that is homologous to a GAVE19 DNA molecule, e.g., is homologous to an isolated nucleic acid molecule having a DNA sequence of SEQ ID NO:1. Two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks using default parameters, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra. Moreover, nucleic acid molecules encoding GAVE19 proteins from other species (GAVE19 homologues) with a nucleotide sequence that differs from that of a human GAVE19, are intended to be within the scope of the invention.

### Variants of an Isolated Nucleic acid Molecule of the present Invention

The present invention further extends to variants of an isolated nucleic acid molecule comprising a DNA sequence of SEQ ID NO:1. Such variants can be degenerate, allelic, or a combination thereof.

Nucleic acid molecules corresponding to natural allelic variants and homologues of the GAVE19 cDNA of the invention can be isolated based on identity with the murine GAVE19 nucleic acids disclosed herein using the murine cDNA or a portion thereof, as a hybridization probe according to standard hybridization techniques under stringent hybridization conditions.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

Moreover, due to degenerate nature of codons in the genetic code, a GAVE19 protein of the present invention can be encoded by numerous isolated nucleic acid molecules. “Degenerate nature” refers to the use of different three-letter codons to specify a particular amino acid pursuant to the genetic code. It is well known in the art that the following codons can be used interchangeably to code for each specific amino acid:

| | | |
|---|---|---|
| Phenylalanine | (Phe or F) | UUU or UUC |
| Leucine | (Leu or L) | UUA or UUG or CUU or CUC or CUA or CUG |
| Isoleucine | (Ile or I) | AUU or AUC or AUA |
| Methionine | (Met or M) | AUG |
| Valine | (Val or V) | GUU or GUC of GUA or GUG |
| Serine | (Ser or S) | UCU or UCC or UCA or UCG or AGU or AGC |
| Proline | (Pro or P) | CCU or CCC or CCA or CCG |
| Threonine | (Thr or T) | ACU or ACC or ACA or ACG |
| Alanine | (Ala or A) | GCU or GCG or GCA or GCG |
| Tyrosine | (Tyr or Y) | UAU or UAC |
| Histidine | (His or H) | CAU or CAC |
| Glutamine | (Gln or Q) | CAA or CAG |
| Asparagine | (Asn or N) | AAU or AAC |
| Lysine | (Lys or K) | AAA or AAG |
| Aspartic Acid | (Asp or D) | GAU or GAC |
| Glutamic Acid | (Glu or E) | GAA or GAG |
| Cysteine | (Cys or C) | UGU or UGC |
| Arginine | (Arg or R) | CGU or CGC or CGA or CGG or AGA or AGG |
| Glycine | (Gly or G) | GGU or GGC or GGA or GGG |
| Tryptophan | (Trp or W) | UGG |
| Termination codon | UAA (ochre) or UAG (amber) or UGA (opal) | |

It should be understood that the codons specified above are for RNA sequences. The corresponding codons for DNA have a T substituted for U.

In addition to the murine GAVE19 nucleotide sequence shown in SEQ ID NO:1, it will be appreciated by those skilled in the art that DNA sequence polymorphisms that lead to changes in the amino acid sequences of GAVE19 may exist within a population. Such genetic polymorphism in the GAVE19 gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a GAVE19 protein, preferably a mammalian GAVE19 protein. As used herein, the phrase “allelic variant” refers to a nucleotide sequence that occurs at a GAVE19 locus or to a polypeptide encoded by the nucleotide sequence. Alternative alleles can be identified by sequencing the gene of interest in a number of different individuals. That can be carried out readily by using hybridization probes to identify the same genetic locus in a variety of individuals. Any and all such nucleotide variations and resulting amino acid polymorphisms or variations in GAVE19 that are the result of natural allelic variation and that do not alter the functional activity of GAVE19 are intended to be within the scope of the invention.

Moreover, variants of an isolated nucleic acid molecule of the present invention can be readily made by one of ordinary skill in the art using routine laboratory techniques, e.g., site-directed mutagenesis.

### Antisense Nucleotide Sequences

The instant invention also extends to antisense nucleic acid molecules, i.e., molecules that are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire GAVE19 coding strand or to only a portion thereof, e.g., all or part of the protein coding region (or open reading frame). An antisense nucleic acid molecule can be antisense to a noncoding region of the coding strand of a nucleotide sequence encoding GAVE19. The noncoding regions (“5′ and 3′ untranslated regions”) are the 5′ and 3′ sequences that flank the coding region and are not translated into amino acids.

Given the coding strand sequences encoding GAVE19 disclosed herein (e.g., SEQ ID NO:1), antisense nucleic acids of the invention can be designed according to the rules of Watson & Crick base pairing. The antisense nucleic acid molecule can be complementary to the entire coding region of GAVE19 mRNA, but more preferably is an oligonucleotide that is antisense to only a portion of the coding or noncoding region of GAVE19 mRNA. For example, the antisense oligonucleotide can be complementary to the region surrounding the translation start site of GAVE19 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be synthesized chemically using naturally occurring nucleotides or various chemically modified nucleotides designed to increase the biological stability of the molecules, or to increase the physical stability of the duplex formed between the antisense and sense nucleic acids, e.g., phosphorothioate derivatives, phosphonate derivatives and acridine-substituted nucleotides can be used.

Examples of modified nucleotides that can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, β,-D-galactosylqueosine, inosine, N⁶-isopentenyladenine, 1-methylguanine, 11-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, β-D-mannosylqueosine, 5-methoxycarboxymethyluracil, 5-methoxyuracil, 2-methylthio-N-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules of the invention typically are administered to a subject or generated in situ so as to hybridize with or bind to cellular mRNA and/or genomic DNA encoding a GAVE19 protein thereby to inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix, or to a regulatory region of GAVE19.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that the molecules specifically bind to receptors or antigens expressed on a selected cell surface, e.g., by linking the antisense nucleic acid molecules to peptides or antibodies that bind to cell surface receptors or antigens. The antisense nucleic acid molecules also can be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in that the strands run parallel to each other (Gaultier et al., Nucleic Acids Res (1987)15:6625-6641). The antisense nucleic acid molecule also can comprise a methylribonucleotide (Inoue et al., Nucleic Acids Res (1987) 15:6131-6148) or a chimeric RNA-DNA analogue (Inoue et al., FEBS Lett (1987) 215:327-330).

### Ribozymes

The invention also encompasses ribozymes. Ribozymes are catalytic RNA molecules with ribonuclease activity that are capable of cleaving a single-stranded nucleic acid, such as an mRNA, that hybridizes to the ribozyme. Thus, ribozymes (e.g., hammerhead ribozymes (described in Haselhoff et al., Nature (1988) 334:585-591)) can be used to cleave catalytically GAVE19 mRNA transcripts, and thus inhibit translation of GAVE19 mRNA. A ribozyme having specificity for a GAVE19-encoding nucleic acid can be designed based on the nucleotide sequence of a GAVE19 DNA disclosed herein (e.g., SEQ ID NO:1). For example, a derivative of a Tetrahymena L-19 IVS RNA can be constructed so that the nucleotide sequence of the active site is complementary to the nucleotide sequence to be cleaved in a GAVE19-encoding mRNA, see, e.g., U.S. Pat. Nos. 4,987,071 and 5,116,742. Alternatively, GAVE19 mRNA can be used to select a catalytic RNA having a specific ribonuclease activity from a pool of RNA molecules, see, e.g., Bartel et al., Science (1993) 261:1411-1418.

### Triple Helical Nucleic Acid Molecules and Peptide Nucleic Acids of the of the Present Invention

The invention also encompasses nucleic acid molecules that form triple helical structures. For example, GAVE19 gene expression can be inhibited by targeting nucleotide sequences complementary to the regulatory region of the GAVE19 (e.g., the GAVE19 promoter and/or enhancers) to form triple helical structures that prevent transcription of the GAVE 19 gene in target cells, see generally, Helene, Anticancer Drug Des (1991) 6(6):569; Helene Ann NY Acad Sci (1992) 660:27; and Maher, Bioassays (1992) 14(12):807.

In particular embodiments, the nucleic acid molecules of the invention can be modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (see Hyrup et al., Bioorganic & Medicinal Chemistry (1996) 4:5). As used herein, the terms “peptide nucleic acids” or “PNAs” refer to nucleic acid mimics, e.g., DNA mimics, in that the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al., Proc Natl Acad Sci USA (1996) 93:14670.

PNAs of GAVE 19 can also be used in therapeutic and diagnostic applications. For example, PNAs can be used as antisense or antigene agents for sequence-specific modulation of gene expression by, e.g., inducing transcription or translation arrest or inhibiting replication. PNAs of GAVE19 also can be used. For example, a PNA can be used in the analysis of single base pair mutations in a gene by, e.g., PNA-directed PCR clamping; as artificial restriction enzymes when used in combination with other enzymes, e.g., S1 nucleases (Hyrup et al. (1996) supra) or as probes or primers for DNA sequence and hybridization (Hyrup et al. (1996) supra; Perry-O'Keefe et al. (1996) supra).

In another embodiment, PNAs of GAVE19 can be modified, e.g., to enhance stability, specificity or cellular uptake, by attaching lipophilic or other helper groups to the PNA, by the formation of PNA-DNA chimeras or by the use of liposomes or other techniques of drug delivery known in the art. The synthesis of PNA-DNA chimeras can be performed as described in Hyrup et al. (1996) supra, Finn et al., Nucleic Acids Res (1996) 24(17):3357-63, Mag et al., Nucleic Acids Res (1989) 17:5973; and Peterser et al., Bioorganic Med Chem Lett (1975) 5:1119.

### GAVE19 Protein

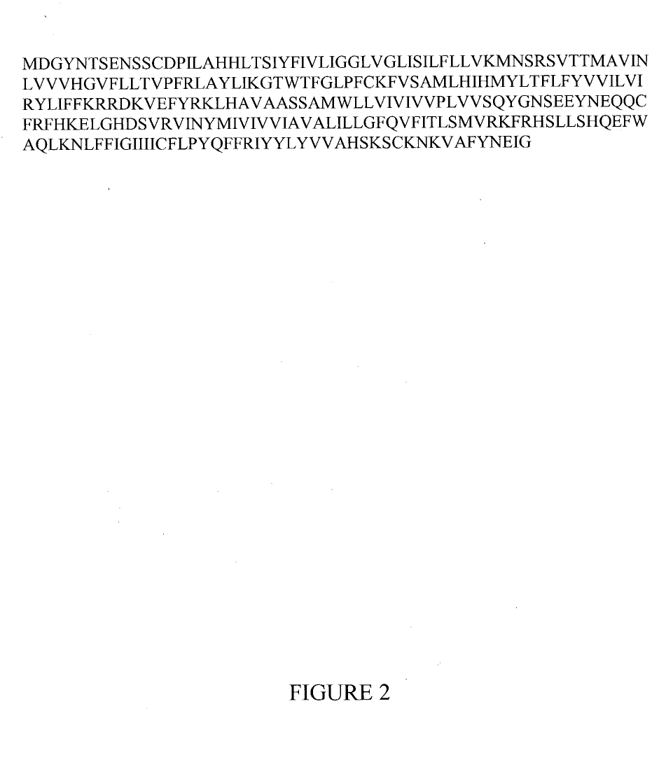

Moreover, the present invention extends to an isolated polypeptide comprising the amino acid sequence of FIG. 2 (SEQ ID NO:2), a variant thereof, a fragment thereof or an analog or derivative thereof.

An isolated nucleic acid molecule encoding a GAVE19 protein having a sequence that differs from that of SEQ ID NO:2, e.g. a variant, can be created by introducing one or more nucleotide substitutions, additions or deletions into the nucleotide sequence of SEQ ID NO:1 such that one or more amino acid substitutions, additions or deletions are introduced into the encoded protein.

In a particular embodiment, a mutant GAVE19 protein can be assayed for: (1) the ability to form protein:protein interactions with proteins in the GAVE19 signaling pathway; (2) the ability to bind a GAVE19 ligand; or (3) the ability to bind to an intracellular target protein. In yet another embodiment, a mutant GAVE 19 can be assayed for the ability to modulate cellular proliferation or cellular differentiation.

Native GAVE19 proteins can be isolated from cells or tissue sources by an appropriate purification scheme using standard protein purification techniques. Alternatively, GAVE19 proteins can readily be produced by recombinant DNA techniques. Yet another alternative encompassed by the present invention is the chemical synthesis of a GAVE19 protein or polypeptide using standard peptide synthesis techniques.

An “isolated” or “purified” protein, or biologically active portion thereof, is substantially free of cellular material or other contaminating proteins from the cell or tissue source from which the GAVE19 protein is derived, or is substantially free of chemical precursors or other chemicals when chemically synthesized. The phrase, “substantially free of cellular material” includes preparations of GAVE19 protein in which the protein is separated from cellular components of the cells from which the protein is isolated or recombinantly produced. Thus, GAVE19 protein that is substantially free of cellular material includes preparations of GAVE19 protein having less than about 30%, 20%, 10% or 5% or less (by dry weight) of non-GAVE19 protein (also referred to herein as a “contaminating protein”). When the GAVE19 protein or biologically active portion thereof is produced recombinantly, it also is preferably substantially free of culture medium, i.e., culture medium represents less than about 20%, 10% or 5% or less of the volume of the protein preparation. When GAVE19 protein is produced by chemical synthesis, it is preferably substantially free of chemical precursors or other chemicals, i.e., it is separated from chemical precursors or other chemicals that are involved in the synthesis of the protein. Accordingly, such preparations of GAVE19 protein have less than about 30%, 20%, 10% or 5% or less (by dry weight) of chemical precursors or non-GAVE19 chemicals.

Biologically active portions or fragments of a GAVE19 protein include peptides comprising amino acid sequences sufficiently identical to or derived from the amino acid sequence of the GAVE19 protein (e.g., the amino acid sequence shown in SEQ ID NO:2), that include fewer amino acids than the full length GAVE19 protein and exhibit at least one activity of a GAVE19 protein. Typically, biologically active portions comprise a domain or motif with at least one activity of a GAVE19 protein. A biologically active portion of a GAVE19 protein can be a polypeptide that is, for example, 10, 25, 50, 100 or more amino acids in length. Particular biologically active polypeptides include one or more identified GAVE19 structural domains.

Moreover, other biologically active portions, in which other regions of the protein are deleted, can be prepared by recombinant techniques and evaluated for one or more of the functional activities of a native GAVE19 protein.

Other useful GAVE 19 proteins are substantially identical to SEQ ID NO:2 and retain a functional activity of the protein of SEQ ID NO:2 yet differ in amino acid sequence due to natural allelic variation or mutagenesis. For example, such GAVE19 proteins and polypeptides possess at least one biological activity described herein.

Accordingly, a useful GAVE19 protein is a protein that includes an amino acid sequence at least about 45%, preferably 55%, 65%, 75%, 85%, 95%, 99% or 100%identical to the amino acid sequence of SEQ ID NO:2 and retains a functional activity of a GAVE19 protein of SEQ ID NO:2. In a particular embodiment, the GAVE19 protein retains a functional activity of the GAVE19 protein of SEQ ID NO:2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions then are compared. When a position in the first sequence is occupied by the same amino acid residue or nucleotide as the corresponding position in the second sequence, then the molecules are considered identical at that position. The percent identity between the two sequences is a function of the number of identical positions shared by the sequences (i.e., percent identity=number of identical positions/total number of positions (e.g., overlapping positions)×100). In one embodiment, the two sequences are the same length.

The determination of percent identity between two sequences can be accomplished using a mathematical algorithm. A particular, non-limiting example of a mathematical algorithm utilized for the comparison of two sequences is the algorithm of Karlin et al., Proc Natl Acad Sci USA (1990) 87:2264, modified as in Karlin et al., Proc Natl Acad Sci USA (1993) 90:5873-5877. Such an algorithm is incorporated into the NBLAST and XBLAST programs of Altschul et al., J Mol Bio (1990) 215:403. BLAST nucleotide searches can be performed with the NBLAST program, for example, score=100, wordlength=12, to obtain nucleotide sequences homologous to a GAVE19 nucleic acid molecule of the present invention. BLAST protein searches can be performed with the XBLAST program, score=50, wordlength=3 to obtain amino acid sequences homologous to a GAVE19 protein molecule of the invention. To obtain gapped alignments for comparison purposes, Gapped BLAST can be utilized as described in Altschul et al., Nucleic Acids Res (1997) 25:3389. Alternatively, PSI-Blast can be used to perform an iterated search that detects distant relationships between molecules. Altschul et al. (1997) supra. When utilizing BLAST, Gapped BLAST and PSI-Blast programs, the default parameters of the respective programs (e.g., XBLAST and NBLAST) can be used, see http://www.ncbi.nlm.nih.gov.

Another particular, non-limiting example of a mathematical algorithm utilized for the comparison of sequences is the algorithm of Myers et al., CABIOS (1988) 4:11-17. Such an algorithm is incorporated into the ALIGN program (version 2.0) that is part of the GCG sequence alignment software package. When utilizing the ALIGN program for comparing amino acid sequences, a PAM120 weight residue table, a gap length penalty of 12 and a gap penalty of 4 may be used.

The percent identity between two sequences can be determined using techniques similar to those described above, with or without allowing gaps. In calculating percent identity, only exact matches are counted.

The present invention further extends to GAVE19 chimeric or fusion proteins. As used herein, a GAVE19 “chimeric protein” or “fusion protein” comprises a GAVE19 polypeptide operably linked to a non-GAVE19 polypeptide. A “GAVE19 polypeptide” refers to a polypeptide having an amino acid sequence corresponding to GAVE19. A “non-GAVE19 polypeptide” refers to a polypeptide having an amino acid sequence corresponding to a protein that is not substantially identical to the GAVE19 protein, e.g., a protein that is different from the GAVE19 protein and is derived from the same or a different organism. Within a GAVE19 fusion protein, the GAVE19 polypeptide can correspond to all or a portion of a GAVE19 protein, preferably at least one biologically active portion of a GAVE19 protein. Within the fusion protein, the term “operably linked” is intended to indicate that the GAVE19 polypeptide and the non-GAVE19 polypeptide are fused in-frame to each other. The non-GAVE19 polypeptide can be fused to the N-terminus or C-terminus of a GAVE19 polypeptide. One useful fusion protein is GST-GAVE19 in which a GAVE19 sequence is fused to the C-terminus of glutathione-S-transferase (GST). Such fusion proteins can facilitate the purification of recombinant GAVE19.

In another embodiment, a fusion protein of the present invention extends to a GAVE19-immunoglobulin fusion protein in which all or part of GAVE19 is fused to sequences derived from a member of the immunoglobulin protein family. The GAVE19-immunoglobulin fusion proteins of the invention can be incorporated into pharmaceutical compositions and administered to a subject to inhibit an interaction between a GAVE19 ligand and a GAVE19 protein on the surface of a cell, thereby to suppress GAVE19-mediated signal transduction in vivo. The GAVE19-immunoglobulin fusion proteins can be used to affect the bioavailability of a GAVE19 cognate ligand. Inhibition of the GAVE19 ligand-GAVE19 interaction may be useful therapeutically, both for treating proliferative and differentiative disorders and for modulating (e.g. promoting or inhibiting) cell survival. Moreover, the GAVE19-immunoglobulin fusion proteins of the invention can be used as immunogens to produce anti-GAVE19 antibodies in a subject, to purify GAVE19 ligands and in screening assays to identify molecules that inhibit the interaction of GAVE19 with a GAVE19 ligand.

In a particular embodiment, a GAVE19 chimeric or fusion protein of the present invention is produced by standard recombinant DNA techniques. For example, DNA fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that subsequently can be annealed and reamplified to generate a chimeric gene sequence (see e.g., Ausubel et al., supra). Moreover, many expression vectors are commercially available that already encode a fusion moiety (e.g., a GST polypeptide). A GAVE19-encoding nucleic acid can be cloned into such an expression vector so that the fusion moiety is linked in-frame to the GAVE19 protein.

### Variants

As explained above, the present invention further extends to variants of the GAVE19 protein. For example, mutations may be introduced into the amino acid sequence of SEQ ID NO:2 using standard techniques, such as site-directed mutagenesis and PCR-mediated mutagenesis. Moreover, conservative amino acid substitutions can be made at one or more predicted non-essential amino acid residues. A “conservative amino acid substitution” is one in which the amino acid residue is replaced with an amino acid residue having a similar side chain. For example, one or more amino acids can be substituted by another amino acid of a similar polarity, which acts as a functional equivalent, resulting in a silent alteration. Substitutes for an amino acid within the amino acid sequence of a polypeptide of the present invention may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to effect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Moreover, amino acid substitutions may also be introduced to substitute an amino acid with a particularly preferable property. For example, a Cys may be introduced for a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Mutations can also be introduced randomly along all or part of a GAVE19 coding sequence, such as by saturation mutagenesis, and the resultant mutants can be screened for GAVE19 biological activity to identify mutants that retain activity. Following mutagenesis, the encoded protein can be expressed recombinantly and the activity of the protein can be determined.

Variants of the present invention can function as a GAVE19 agonist (mimetic) or as GAVE19 antagonist. Variants of the GAVE19 protein can be generated by mutagenesis, e.g., discrete point mutation or truncation of the GAVE19 protein. An agonist of the GAVE19 protein can retain substantially the same or a subset of the biological activities of the naturally occurring GAVE19 protein. For example, an antagonist of the GAVE19 protein can competitively bind to a downstream or upstream member of a cellular signaling cascade that includes the GAVE19 protein, and thus inhibit one or more of the activities of the naturally occurring form of the GAVE19 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the GAVE 19 proteins.

Variants of the GAVE19 protein that function as either GAVE19 agonists (mimetics) or as GAVE19 antagonists can be identified by screening combinatorial libraries of mutants, e.g., truncation mutants, of the GAVE19 protein for GAVE19 agonist or antagonist activity. In one embodiment, a variegated library of GAVE19 variants is generated by combinatorial mutagenesis at the nucleic acid level, and is encoded by a variegated gene library. A variegated library of GAVE19 variants can be produced by, for example, enzymatically ligating a mixture of synthetic oligonucleotides into gene sequences such that a degenerate set of potential GAVE19 sequences is expressed as individual polypeptides or alternatively, as a set of larger fusion proteins (e.g., for phage display) containing the set of GAVE19 sequences therein. There are a variety of methods that can be used to produce libraries of potential GAVE19 variants from a degenerate oligonucleotide sequence. Chemical synthesis of a degenerate gene sequence can be performed in an automated DNA synthesizer and the synthetic gene then ligated into an appropriate expression vector. Use of a degenerate set of genes allows for the provision, in one mixture, of all of the sequences encoding the desired set of potential GAVE19 sequences. Methods for synthesizing degenerate oligonucleotides are known in the art (see, e.g., Narang, Tetrahedron (1983) 39:3; Itakura et al., Ann Rev Biochem (1984) 53:323; Itakura et al., Science (1984) 198:1056; Ike et al., Nucleic Acid Res (1983) 11:477).

In addition, libraries of fragments of the GAVE19 protein coding sequence can be used to generate a variegated population of GAVE19 fragments for screening and subsequent selection of variants of a GAVE19 protein. In one embodiment, a library of coding sequence fragments can be generated by treating a double-stranded PCR fragment of a GAVE19 coding sequence with a nuclease under conditions wherein nicking occurs only about once per molecule, denaturing the double-stranded DNA, renaturing the DNA to form double-stranded DNA that can include sense/antisense pairs from different nicked products, removing single-stranded portions from reformed duplexes by treatment with S1 nuclease and ligating the resulting fragment library into an expression vector. By this method, an expression library can be derived that encodes N-terminal and internal fragments of various sizes of the GAVE19 protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of GAVE19 proteins. The most widely used techniques that are amenable to high through-put analysis for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify GAVE19 variants (Arkin et al., Proc Natl Acad Sci USA (1992) 89:7811-7815; Delgrave et al., Protein Engineering (1993) 6(3):327-331).

### Analogs and Derivatives of GAVE19

Moreover, the present invention also includes derivatives or analogs of GAVE19 produced from a chemical modification. A GAVE19 protein of the present invention may be derivatized by the attachment of one or more chemical moieties to the protein moiety.

Chemical Moieties For Derivatization. The chemical moieties suitable for derivatization may be selected from among water soluble polymers so that the GAVE19 analog or derivative does not precipitate in an aqueous environment, such as a physiological environment. Optionally, the polymer will be pharmaceutically acceptable. One skilled in the art will be able to select the desired polymer based on such considerations as whether the polymer/component conjugate will be used therapeutically, and if so, the desired dosage, circulation time, resistance to proteolysis, and other considerations. For GAVE19, these may be ascertained using the assays provided herein. Examples of water soluble polymers having applications herein include, but are not limited to, polyethylene glycol, copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), dextran, poly(n-vinyl pyrrolidone)polyethylene glycol, propropylene glycol homopolymers, polypropylene oxide/ethylene oxide co-polymers, polyoxyethylated polyols or polyvinyl alcohol. Polyethylene glycol propionaldenhyde may have advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects if any, on biological activity, the ease in handling, the degree or lack of antigenicity and other known effects of the polyethylene glycol to a therapeutic protein or analog).

The number of polymer molecules so attached to GAVE19 may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to GAVE19 molecules will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

The polyethylene glycol molecules (or other chemical moieties) should be attached to GAVE19 with consideration of effects on functional or antigenic domains of GAVE19. There are a number of attachment methods available to those skilled in the art, e.g., EP 0 401 384 herein incorporated by reference (coupling PEG to G-CSF), see also Malik et al., 1992, Exp. Hematol. 20:1028-1035 (reporting pegylation of GM-CSF using tresyl chloride). For example, polyethylene glycol may be covalently bound through amino acid residues via a reactive group, such as a free amino or carboxyl group. Reactive groups are those to which an activated polyethylene glycol molecule may be bound. The amino acid residues having a free amino group include lysine residues and the N-terminal amino acid residues; those having a free carboxyl group include aspartic acid residues, glutamic acid residues and the C-terminal amino acid residue. Sulfhydryl groups may also be used as a reactive group for attaching the polyethylene glycol molecule(s). Preferred for therapeutic purposes is attachment at an amino group, such as attachment at the N-terminus or lysine group.

One may specifically desire N-terminally chemically modified GAVE19. Using polyethylene glycol as an illustration of the present compositions, one may select from a variety of polyethylene glycol molecules (by molecular weight, branching, etc.), the proportion of polyethylene glycol molecules to GAVE19 molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemical modification may be accomplished by reductive alkylation which exploits differential reactivity of different types of primary amino groups (lysine versus the N-terminal) available for derivatization in GAVE19. Under the appropriate reaction conditions, substantially selective derivatization of GAVE19 at the N-terminus with a carbonyl group containing polymer is achieved. For example, one may selectively N-terminally pegylate GAVE19 by performing the reaction at a pH which allows one to take advantage of the pKₐ differences between the ε-amino groups of the lysine residues and that of the α-amino group of the N-terminal residue of GAVE19. By such selective derivatization, attachment of a water soluble polymer to GAVE19 is controlled: the conjugation with the polymer takes place predominantly at the N-terminus of GAVE19 and no significant modification of other reactive groups, such as the lysine side chain amino groups, occurs. Using reductive alkylation, the water soluble polymer may be of the type described above, and should have a single reactive aldehyde for coupling to GAVE19. Polyethylene glycol proprionaldehyde, containing a single reactive aldehyde, may be used.

### Antibodies of GAVE19, Variants Thereof, Fragments Thereof, or Analogs or Derivatives Thereof

An isolated GAVE19 protein or a portion or fragment thereof, can be used as an immunogen to generate antibodies that bind GAVE19 using standard techniques for polyclonal and monoclonal antibody preparation. The term “antibody” as used herein refers to immunoglobulin molecules and immunologically active portions of immunoglobulin molecules, i.e., molecules that contain an antigen-binding site that specifically binds an antigen, such as GAVE19, or a fragment thereof. A molecule that specifically binds to GAVE19 is a molecule that binds GAVE19, but does not substantially bind other molecules in a sample, e.g., a biological sample that naturally contains GAVE19. Examples of immunologically active portions of immunoglobulin molecules include F^{(ab)} and F^{(ab′)2} fragments that can be generated by treating the antibody with an enzyme such as pepsin. The invention provides polyclonal, monoclonal and chimeric antibodies that have GAVE19, a variant thereof, a fragment thereof, or an analog or derivative thereof, as an immunogen.

The full-length GAVE19 protein can be used or, alternatively, the invention provides antigenic peptide fragments of GAVE19 for use as immunogens. The antigenic peptide of GAVE19 comprises at least 8 (preferably 10, 15, 20, 30 or more) amino acid residues of the amino acid sequence shown in SEQ ID NO:2 and encompasses an epitope of GAVE19 such that an antibody raised against the peptide forms a specific immune complex with GAVE19.

A GAVE19 immunogen typically is used to prepare antibodies by immunizing a suitable subject, (e.g., rabbit, goat, mouse or other mammal) with the immunogen. An appropriate immunogenic preparation can contain, for example, recombinantly expressed GAVE19 protein or a chemically synthesized GAVE19 polypeptide. The preparation further can include an adjuvant, such as Freund's complete or incomplete adjuvant or similar immunostimulatory agent. Immunization of a suitable subject with an immunogenic GAVE19 preparation induces a polyclonal anti-GAVE19 antibody response.

An antibody of the present invention can be a monoclonal antibody, a polyclonal antibody, or a chimeric antibody. The term “monoclonal antibody” or “monoclonal antibody composition”, as used herein, refers to a population of antibody molecules that contain only one species of an antigen-binding site capable of immunoreacting with a particular epitope of GAVE19. A monoclonal antibody composition thus typically displays a single binding affinity for a particular GAVE19 protein epitope.

Polyclonal anti-GAVE19 antibodies can be prepared as described above by immunizing a suitable subject with a GAVE19 immunogen. The anti-GAVE19 antibody titer in the immunized subject can be monitored over time by standard techniques, such as with an enzyme-linked immunosorbent assay (ELISA) using immobilized GAVE19. If desired, the antibody molecules directed against GAVE19 can be isolated from the mammal (e.g., from the blood) and further purified by well-known techniques, such as protein A chromatography, to obtain the IgG fraction. At an appropriate time after immunization, e.g., when the anti-GAVE19 antibody titers are highest, antibody-producing cells can be obtained from the subject and used to prepare monoclonal antibodies by standard techniques, such as the hybridoma technique originally described by Kohler et al., Nature (1975) 256:495-497, the human B cell hybridoma technique (Kohler et al., Immunol Today (1983) 4:72), the EBV hybridoma technique (Cole et al., Monoclonal Antibodies and Cancer Therapy, (1985), Alan R. Liss, Inc., pp. 77-96) or trioma techniques. The technology for producing hybridomas is well known (see generally Current Protocols in Immunology (1994) Coligan et al., eds., John Wiley & Sons, Inc., New York, N.Y.). Briefly, an immortal cell line (typically a myeloma) is fused to lymphocytes (typically splenocytes) from a mammal immunized with a GAVE19 immunogen as described above and the culture supernatants of the resulting hybridoma cells are screened to identify a hybridoma producing a monoclonal antibody that binds GAVE19.

Any of the many well known protocols used for fusing lymphocytes and immortalized cell lines can be applied for the purpose of generating an anti-GAVE19 monoclonal antibody (see, e.g., Current Protocols in Immunology, supra; Galfre et al., Nature (1977) 266:550-552; Kenneth, in Monoclonal Antibodies: A New Dimension In Biological Analyses, Plenum Publishing Corp., New York, N.Y. (1980); and Lerner, Yale J Biol Med (1981) 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the instant invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. The myeloma lines are available from ATCC. Typically, HAT-sensitive mouse myeloma cells are fused to mouse splenocytes using polyethylene glycol (“PEG”). Hybridoma cells resulting from the fusion then are selected using HAT medium that kills unfused and unproductively fused myeloma cells (unfused splenocytes die after several days because they are not transformed). Hybridoma cells producing a monoclonal antibody of the invention are detected by screening the hybridoma culture supernatants for antibodies that bind GAVE19, e.g., using a standard ELISA assay.

Alternative to preparing monoclonal antibody-secreting hybridomas, a monoclonal anti-GAVE19 antibody can be identified and isolated by screening a recombinant combinatorial immunoglobulin library (e.g., an antibody phage display library) with GAVE19 thereby to isolate immunoglobulin library members that bind GAVE19. Kits for generating and screening phage display libraries are commercially available (e.g., the Pharmacia Recombinant Phage Antibody System, Catalog No. 27-9400-01; and the Stratagene “SURFZAP” Phage Display Kit, Catalog No. 240612).

Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology (1991) 9:1370-1372; Hay et al., Hum Antibody Hybridomas (1992) 3:81-85; Huse et al., Science (1989) 246:1275-1281; and Griffiths et al., EMBO J (1993) 25(12):725-734.

Furthermore, recombinant anti-GAVE19 antibodies, including, e.g., monoclonal and chimeric antibodies, can be made using standard recombinant DNA techniques. Such antibodies can be produced by recombinant DNA techniques known in the art, for example using methods described in PCT Publication No. WO 87/02671; Europe Patent Application No. 184,187; Europe Patent Application No. 171,496; Europe Patent Application No. 173,494; PCT Publication No. WO 86/01533; U.S. Pat. No. 4,816,567; Europe Patent Application No. 125,023; Better et al., Science (1988) 240:1041-1043; Liu et al., Proc Natl Acad Sci USA (1987) 84:3439-3443; Lin et al., J Immunol (1987) 139:3521-3526; Sun et al., Proc Natl Acad Sci USA (1987) 84:214-218; Nishimura et al., Canc Res (1987) 47:999-1005; Wood et al., Nature (1985) 314:446-449; Shaw et al., J Natl Cancer Inst (1988) 80:1553-1559; Morrison, Science (1985) 229:1202-1207; Oi et al., Bio/Techniques (1986) 4:214; U.S. Pat. No. 5,225,539; Jones et al., Nature (1986) 321:552-525; Verhoeyan et al., Science (1988) 239:1534; and Beidler et al., J Immunol (1988) 141:4053-4060.

An anti-GAVE19 antibody (e.g., monoclonal antibody) can be used to isolate GAVE19 by standard techniques, such as affinity chromatography or immunoprecipitation. An anti-GAVE19 antibody can facilitate the purification of natural GAVE19 from cells and of recombinantly produced GAVE19 expressed in host cells. Moreover, an anti-GAVE19 antibody can be used to detect GAVE19 protein (e.g., in a cellular lysate or cell supernatant) to evaluate the abundance and pattern of expression of the GAVE19 protein. Anti-GAVE19 antibodies can be used diagnostically to monitor protein levels in tissue as part of a clinical testing procedure, for example, to determine the efficacy of a given treatment regimen. Detection can be facilitated by coupling the antibody to a detectable substance, which are described infra.

### Detectable Labels

Optionally, isolated nucleic acid molecules of the present invention, polypeptides of the present invention, and antibodies of the present invention, as well as fragments of such moieties, may be detectably labeled. Suitable labels include enzymes, fluorophores (e.g., fluorescene isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), bioluminescent materials, and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized calorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labelling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70. 419-439, 1980 and in U.S. Pat. No. 4,857,453.

Other labels for use in the invention include magnetic beads or magnetic resonance imaging labels.

In another embodiment, a phosphorylation site can be created on an isolated polypeptide of the present invention, an antibody of the present invention, or a fragment thereof, for labeling with ³²P, e.g., as described in European Patent No. 0372707 or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

### Recombinant Expression Vectors and Host Cells

Another aspect of the invention pertains to vectors, preferably expression vectors, containing a nucleic acid encoding GAVE19 (or a portion thereof). As explained above, one type of vector is a “plasmid,” which refers to a circular double-stranded DNA loop into which additional DNA segments can be ligated. Another type of vector is a viral vector, wherein additional DNA segments can be ligated into a viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell on introduction into the host cell and thereby are replicated along with the host genome. Moreover, expression vectors are capable of directing the expression of genes operably linked thereto. In general, expression vectors of utility in recombinant DNA techniques are often in the form of plasmids (vectors). However, the invention is intended to include other forms of expression vectors, such as viral vectors (e.g., replication defective retroviruses, adenoviruses and adeno-associated viruses), that serve equivalent functions.

A recombinant expression vector of the invention comprises a nucleic acid molecule of the present invention in a form suitable for expression of the nucleic acid in a host cell. That means a recombinant expression vector of the present invention includes one or more regulatory sequences, selected on the basis of the host cells to be used for expression, that is operably linked to the nucleic acid to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology Vol. 185, Academic Press, San Diego, Calif. (1990). Regulatory sequences include those that direct constitutive expression of the nucleotide sequence in many types of host cells (e.g., tissue specific regulatory sequences). It will be appreciated by those skilled in the art that the design of the expression vector can depend on such factors as the choice of host cell to be transformed, the level of expression of protein desired etc. The expression vectors of the invention can be introduced into host cells to produce proteins or peptides encoded by nucleic acids as described herein (e.g., GAVE19 proteins, mutant forms of GAVE19, fusion proteins etc.).

A recombinant expression vector of the invention can be designed for expression of GAVE19 in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using phage regulatory elements and proteins, such as, a T7 promoter and/or a T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes and the cognate recognition sequences, include Factor Xa, thrombin and enterokinase. Typical fusion expression vectors include pGEX (Pharmacia Biotech Inc; Smith et al., Gene (1988) 67:31-40), pMAL (New England Biolabs, Beverly, Mass.) and pRITS (Pharmacia, Piscataway, N.J.), that fuse glutathione 5-transferase (GST), maltose E binding protein or protein A, respectively, to the target recombinant protein.

Examples of suitable inducible non-fusion E. coli expression vectors include pTrc (Amann et al., Gene (1988) 69:301-315) and pET 11d (Studier et al., Gene Expression Technology: Methods in Enzymology, Academic Press, San Diego, Calif. (1990) 185:60-89). Target gene expression from the pTrc vector relies on host RNA polymerase transcription from a hybrid trp-lac fusion promoter.

One strategy to maximize recombinant protein expression in E. coli is to express the protein in a host with impaired capacity to cleave proteolytically the recombinant protein (Gottesman, Gene Expression Technology: Methods in Enzymology, Academic Press, San Diego, Calif. (1990) 185:119-128). Another strategy is to alter the nucleic acid sequence of the nucleic acid molecule to be inserted into an expression vector so that the individual codons for each amino acid are those preferentially utilized in E. coli (Wada et al., Nucleic Acids Res (1992) 20:2111-2118). Such alteration of nucleic acid sequences of the invention can be carried out by standard DNA synthesis techniques.

In another embodiment, the GAVE19 expression vector is a yeast expression vector. Examples of vectors for expression in yeast such as S. cerevisiae include pYepSecl (Baldari et al., EMBO J (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.).

Alternatively, GAVE19 can be expressed in insect cells using baculovirus expression vectors. Baculovirus vectors available for expression of proteins in cultured insect cells (e.g., Sf9 cells) include the pAc series (Smith et al., Mol Cell Biol (1983) 3:2156-2165) and the pVL series (Lucklow et al., Virology (1989) 170:31-39).

In yet another embodiment, a nucleic acid of the invention is expressed in mammalian cells using a mammalian expression vector. Examples of mammalian expression vectors having applications herein include, but certainly are not limited to pCDM8 (Seed, Nature (1987) 329:840) and pMT2PC (Kaufman et al., EMBO J (1987) 6:187-195). When used in mammalian cells, control functions of the expression vector often are provided by viral regulatory elements. For example, commonly used promoters are derived from polyoma, adenovirus 2, cytomegalovirus and simian virus 40. For other suitable expression systems for both prokaryotic and eukaryotic cells, see chapters 16 and 17 of Sambrook et al., supra.

In another embodiment, a recombinant mammalian expression vector of the present invention is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev (1987) 1:268-277), lymphoid-specific promoters (Calame et al., Adv Immunol (1988) 43:235-275), in particular, promoters of T cell receptors (Winoto et al., EMBO J (1989) 8:729-733) and immunoglobulins (Banerji et al., Cell (1983) 33:729-740; Queen et al., Cell (1983) 33:741-748), neuron-specific promoters (e.g., the neurofilament promoter; Byrne et al., Proc Natl Acad Sci USA (1989) 86:5473-5477), pancreas-specific promoters (Edlund et al., Science (1985) 230:912-916) and mammary gland-specific promoters (e.g., milk whey promoter; U.S. Pat. No. 4,873,316 and Europe Application No. 264,166). Developmentally-regulated promoters also are encompassed, for example the murine hox promoters (Kessel et al., Science (1990) 249:374-379) and the α-fetoprotein promoter (Campes et al., Genes Dev (1989) 3:537-546).

The invention further provides a recombinant expression vector comprising a DNA molecule of the invention cloned into an expression vector in an antisense orientation. That is, the DNA molecule is operably linked to a regulatory sequence in a manner that allows for expression (by transcription of the DNA molecule) of an RNA molecule that is antisense to GAVE19 mRNA. Regulatory sequences operably linked to a nucleic acid cloned in the antisense orientation can be chosen that direct the continuous expression of the antisense RNA molecule in a variety of cell types. For example, viral promoters and/or enhancers or regulatory sequences can be chosen that direct constitutive, tissue-specific or cell type-specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (Reviews-Trends in Genetics, Vol. 1(1)1986).

Another aspect of the present invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but still are included within the scope of the term as used herein.

A host cell can be any prokaryotic or eukaryotic cell. For example, GAVE19 protein can be expressed in bacterial cells such as E. coli, insect cells, yeast or mammalian cells (such as Chinese hamster ovary cells (CHO), 293 cells or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction, DEAE-dextran-mediated transfection, lipofection or electroporation.

For stable transfection of mammalian cells, it is known that, depending on the expression vector and transfection technique used, only a small fraction of cells may integrate the foreign DNA into the genome. To identify and to select the integrants, a gene that encodes a selectable marker (e.g., for resistance to antibiotics) generally is introduced into the host cells along with the gene of interest. Preferred selectable markers include those that confer resistance to drugs, such as G418, hygromycin and methotrexate. Nucleic acid encoding a selectable marker can be introduced into a host cell on the same vector as that encoding GAVE19 or can be introduced on a separate vector. Cells stably transfected with the introduced nucleic acid can be identified by drug selection (e.g., cells that have incorporated the selectable marker gene will survive, while the other cells die).

A host cell of the present invention, such as a prokaryotic or eukaryotic host cell in culture, can be used to produce (i.e., express) GAVE19 protein. Accordingly, the invention further provides methods for producing GAVE19 protein using the host cells of the invention. In one embodiment, the method comprises culturing a host cell of the present invention (into which a recombinant expression vector encoding GAVE19 has been introduced) in a suitable medium such that GAVE19 protein is produced. In another embodiment, the method further comprises isolating GAVE19 from the medium or the host cell.

In another embodiment, GAVE19 comprises an inducible expression system for the recombinant expression of other proteins subcloned in modified expression vectors. For example, host cells comprising a mutated G protein (e.g., yeast cells, Y2 adrenocortical cells and cyc⁻ S49, see U.S. Pat. No. 6,168,927 B1, U.S. Pat. Nos. 5,739,029 and 5,482,835; Mitchell et al., Proc Natl Acad Sci USA (1992) 89(19):8933-37 and Katada et al., J Biol Chem (1984) 259(6):3586-95) are transduced with a first expression vector comprising a nucleic acid sequence encoding GAVE19, wherein GAVE19 is functionally expressed in the host cells. Even though the expressed GAVE19 is constitutively active, the mutation does not allow for signal transduction; i.e., no activation of a G-protein directed downstream cascade occurs (e.g., no adenylyl cyclase activation). Subsequently, a second expression vector is used to transduce the GAVE19-comprising host cells. The second vector comprises a structural gene that complements the G protein mutation of the host cell (i.e., functional mammalian or yeast Gₛ, Gᵢ, Gₒ, or G_{q}, e.g., see PCT Publication No. WO 97/48820; U.S. Pat. Nos. 6,168,927 B1, U.S. Pat. No. 5,739,029 and 5,482,835) in addition to the gene of interest to be expressed by the inducible system. The complementary structural gene of the second vector is inducible; i.e., under the control of an exogenously added component (e.g., tetracycline, IPTG, small molecules etc., see Sambrook et al. supra) that activates a promoter which is operably linked to the complementary structural gene. On addition of the inducer, the protein encoded by the complementary structural gene is functionally expressed such that the constitutively active GAVE19 now will form a complex that leads to appropriate downstream pathway activation (e.g., second messenger formation). The gene of interest comprising the second vector possesses an operably linked promoter that is activated by the appropriate second messenger (e.g., CREB, AP1 elements). Thus, as second messenger accumulates, the promoter upstream from the gene of interest is activated to express the product of said gene. When the inducer is absent, expression of the gene of interest is switched off.

In a particular embodiment, the host cells for the inducible expression system include, but are not limited to, S49 (cyc⁻) cells. While cell lines are contemplated that comprise G-protein mutations, suitable mutants may be artificially produced/constructed (see U.S. Pat. Nos. 6,168,927 B1,U.S. Pat. Nos. 5,739,029 and 5,482,835 for yeast cells).

In a related aspect, the cells are transfected with a vector operably linked to a cDNA comprising a sequence encoding a protein as set forth in SEQ ID NO:2. The first and second vectors comprising said system are contemplated to include, but are not limited to, pCDM8 (Seed, Nature (1987) 329:840) and pMT2PC (Kaufman et al., EMBO J (1987) 6:187-195), pYepSecl (Baldari et al., EMBO J (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.). In a related aspect, the host cells may be transfected by such suitable means, wherein transfection results in the expression of a functional GAVE19 protein (e.g., Sambrook et al., supra, and Kriegler, Gene Transfer and Expression: A Laboratory Manual, Stockton Press, New York, N.Y., 1990). Such “functional proteins” include, but are not limited to, proteins that once expressed, form complexes with G-proteins, where the G-proteins regulate second messenger formation. Other methods for transfecting host cells that have applications herein include, but certainly are not limited to transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

A large variety of promoters have applications in the present invention. Indeed, expression of a polypeptide of the present invention may be controlled by any promoter/enhancer element known in the art, but these regulatory elements must be functional in the host selected for expression. Promoters which may be used to control GAVE19 expression include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Expression vectors containing a nucleic acid molecule of the invention can be identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding GAVE19 protein, a variant thereof, or an analog or derivative thereof, is inserted within the “selection marker” gene sequence of the vector, recombinants containing the insert can be identified by the absence of the GAVE19 gene function. In the fourth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988, Gene 67:31-40), pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamHl cloning site; Summers), pVL1393 (BamHl, SmaI, XbaI, EcoRl, NotI, XmaIII, BglII, and PstI cloning site; Invitrogen), pVL1392 (BglII, PstI, NotI, XmaIII, EcoRI, XbaI, SmaI, and BamHl cloning site; Summers and Invitrogen), and pBlueBacIII (BamHl, BglII, PstI, NcoI, and HindIII cloning site, with blue/white recombinant screening possible; Invitrogen), and fusion transfer vectors, such as but not limited to pAc700 (BamHl and KpnI cloning site, in which the BamHl recognition site begins with the initiation codon; Summers), pAc701 and pAc702 (same as pAc700, with different reading frames), pAc360 (BamHl cloning site 36 base pairs downstream of a polyhedrin initiation codon; Invitrogen(195)), and pBlueBacHisA, B, C (three different reading frames, with BamHl, BglII, PstI, NcoI, and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamHl, SfiI, XhoI, NotI, NheI, HindIII, NheI, PvuII, and KpnI cloning site, constitutive RSV-LTR promoter, hygromycin selectable marker; Invitrogen), pCEP4 (BamHl, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamHl cloning site, inducible metallothionein Iia gene promoter, hygromycin selectable marker: Invitrogen), pREP8 (BamHl, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindII, SbaI, BamHI, and Hpa cloning site, TK or XPRT selection).

Yeast expression systems can also be used according to the invention to express GAVE19 protein, a variant thereof, or an analog or derivative thereof. For example, the non-fusion pYES2 vector (XbaI, SphI, ShoI, NotI, GstXI, EcoRI, BstXI, BamHl, Sac, Kpnl, and HindIII cloning sit; Invitrogen) or the fusion pYESHisA, B, C (XbaI, SphI, ShoI, NotI, BstXI, EcoRI, BamHl, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors that can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification (e.g., glycosylation, cleavage [e.g., of signal sequence]) of proteins. Appropriate cell lines or host systems can be chosen to ensure the desired modification and processing of the foreign protein expressed. For example, expression in a bacterial system can be used to produce an nonglycosylated core protein product.

### Transgenic Animals

A host cell of the present invention also can be used to produce transgenic animals. For example, in one embodiment, a host cell of the invention is a fertilized oocyte or an embryonic stem cell into which GAVE19-coding sequences have been introduced. Such host cells then can be used to create non-human transgenic animals into which exogenous GAVE19 sequences have been introduced into the genome, or homologous recombinant animals in which endogenous GAVE19 sequences have been altered. Such animals are useful for studying the function and/or activity of GAVE19 and for identifying and/or evaluating modulators of GAVE19 activity. As used herein, a “transgenic animal” is a non-human animal, preferably a mammal, in which one or more of the cells of the animal includes a transgene. Examples of transgenic animals include non-human primates, sheep, dogs, cows, goats, chickens, rats, amphibians etc.

As used herein, the term “transgene” refers to exogenous DNA that is integrated into the genome of a cell from which a transgenic animal develops and that remains in the genome of the mature animal. The transgene directs the expression of an encoded gene product in one or more cell types or tissues of the transgenic animal. As used herein, a “homologous recombinant animal” is a non-human animal, preferably a mammal, in which an endogenous GAVE19 gene has been altered by homologous recombination. That is accomplished between the endogenous gene and an exogenous DNA molecule introduced into a cell of the animal, e.g., an embryonic cell of the animal, prior to development of the animal.

A transgenic animal of the invention can be created by introducing a GAVE19-encoding nucleic acid molecule into the male pronuclei of a fertilized oocyte using one of the transfection methods described above. The oocyte is then allowed to develop in a pseudopregnant female foster animal. The GAVE19 cDNA sequence e.g., that of (SEQ ID NO:1), for example, can be introduced as a transgene into the genome of a non-human animal. Intronic sequences and polyadenylation signals also can be included in the transgene to increase the efficiency of expression of the transgene. A tissue-specific regulatory sequence(s) can be operably linked to the GAVE19 transgene to direct expression of GAVE19 protein in particular cells. Methods for generating transgenic animals via embryo manipulation and microinjection are conventional in the art and are described, for example, in U.S. Pat. Nos. 4,736,866 and 4,870,009 and U.S. Pat. No. 4,873,191. Similar methods are used for production of other transgenic animals with a transgene in the genome and/or expression of GAVE19 mRNA in tissues or cells of the animals. A transgenic founder animal then can be used to breed additional animals carrying the transgene. Moreover, transgenic animals carrying a transgene encoding GAVE19 can be bred further to other transgenic animals carrying other transgenes.

To create a homologous recombinant animal, a vector is prepared that contains at least a portion of a GAVE19 gene into which a deletion, addition or substitution has been introduced thereby to alter, e.g., functionally disrupt, the GAVE19 gene. In a particular embodiment, the vector is designed such that, on homologous recombination, the endogenous GAVE19 gene is disrupted functionally (i.e., no longer encodes a functional protein; also referred to as a “knock out” vector).

Alternatively, the vector can be designed such that, on homologous recombination, the endogenous GAVE19 gene is mutated or otherwise altered but still encodes functional protein (e.g., the upstream regulatory region can be altered thereby to alter the expression of the endogenous GAVE19 protein).

In the homologous recombination vector, the altered portion of the GAVE19 gene is flanked at the 5′ and 3′ ends by an additional nucleic acid sequence of the GAVE19 gene to allow for homologous recombination to occur between the exogenous GAVE19 gene carried by the vector and an endogenous GAVE19 gene in an embryonic stem cell. The additional flanking GAVE19 nucleic acid sequence is of sufficient length for successful homologous recombination with the endogenous gene. Typically, several kilobases of flanking DNA (both at the 5′ and 3′ ends) are included in the vector (see, e.g., Thomas et al., Cell (1987) 51:503 for a description of homologous recombination vectors).

The vector is introduced into an embryonic stem cell line (e.g., by electroporation) and cells in which the introduced GAVE19 gene has homologously recombined with the endogenous GAVE19 gene are selected (see, e.g., Li et al., Cell (1992) 69:915). The selected cells then are injected into a blastocyst of an animal to form aggregation chimeras (see, e.g., Bradley in Teratocarcinomas and Embryonic Stem Cells: A Practical Approach, Robertson, ed., IRL, Oxford, (1987) pp. 113-152). A chimeric embryo then can be implanted into a suitable pseudopregnant female foster animal and the embryo brought to term. Progeny harboring the homologously recombined DNA in the germ cells can be used to breed animals wherein all cells of the animal contain the homologously recombined DNA by germline transmission of the transgene.

Methods for constructing homologous recombination vectors and homologous recombinant animals are described further in Bradley, Current Opinion in Bio/Technology (1991) 2:823-829 and in PCT Publication Nos. WO 90/11354, WO 91/01140, WO 92/0968 and WO 93/04169.

In another embodiment, transgenic non-human animals can be produced that contain selected systems to allow for regulated expression of the transgene. One example of such a system is the cre/loxP recombinase system of bacteriophage P1. For a description of the cre/loxP recombinase system, see, e.g., Lakso et al., Proc Natl Acad Sci USA (1992) 89:6232-6236. Another example of a recombinase system is the FLP recombinase system of S. cerevisiae (O'Gorman et al., Science (1991) 251:1351-1355). If a cre/loxP recombinase system is used to regulate expression of the transgene, animals containing transgenes encoding both the cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein also can be produced according to the methods described in Wilmut et al., Nature (1997) 385:810-813 and PCT Publication Nos. WO 97/07668 and WO 97/07669. In brief, a cell, e.g., a somatic cell, from the transgenic animal can be isolated and induced to exit the growth cycle and enter Go phase. The quiescent cell then can be fused, e.g., through the use of electrical pulses, to an enucleated oocyte from an animal of the same species from which the quiescent cell is isolated. The reconstructed oocyte then is cultured such that it develops to morula or blastocyte, and then is transferred to a pseudopregnant female foster animal. The offspring borne of the female foster animal will be a clone of the animal from that the cell, e.g., the somatic cell, is isolated.

### Uses and Methods of the Invention

The nucleic acid molecules, proteins, protein homologues, antibodies of the present invention, and fragments of such moieties, may be used in one or more of the following methods: a) screening assays; b) detection assays (e.g., chromosomal mapping, tissue typing, forensic biology); c) predictive medicine (e.g., diagnostic assays, prognostic assays, monitoring clinical trials and pharmacogenomics); and d) methods of treatment (e.g., therapeutic and prophylactic). A GAVE19 protein interacts with other cellular proteins, and thus can be used for (i) regulation of cellular proliferation; (ii) regulation of cellular differentiation; and (iii) regulation of cell survival. The isolated nucleic acid molecules of the invention can be used to express GAVE19 protein (e.g., via a recombinant expression vector in a host cell in gene therapy applications), to detect GAVE19 mRNA (e.g., in a biological sample) or to detect a genetic lesion in a GAVE19 gene and to modulate GAVE19 activity. In addition, a GAVE19 protein can be used to screen drugs or compounds that modulate GAVE19 activity or expression. Such drugs or compounds may readily have applications in treating diseases inflammatory diseases such as rheumatoid arthritis, COPD, etc. Screening for the production of GAVE19 protein forms that have decreased or aberrant activity compared to GAVE19 wild type protein can also be performed with the present invention. In addition, an anti-GAVE 19 antibody of the invention can be used to detect and to isolate GAVE19 proteins and to modulate GAVE19 activity. The invention further pertains to novel agents identified by the above-described screening assays and uses thereof for treatments as described herein.

Screening Assays

Activation of a G protein receptor in the presence of endogenous ligand allows for G protein receptor complex formation, thereupon leading to the binding of GTP to the G protein. The GTPase domain of the G protein slowly hydrolyzes the GTP to GDP resulting, under normal conditions, in receptor deactivation. However, constitutively activated receptors continue to hydrolyze GDP to GTP.

A non-hydrolyzable substrate of G protein, [³⁵S]GTPγS, can be used to monitor enhanced binding to membranes which express constitutively activated receptors. Traynor and Nahorski reported that [³⁵S]GTPγS can be used to monitor G protein coupling to membranes in the absence and presence of ligand (Traynor et al., Mol Pharmacol (1995) 47(4):848-54). A preferred use of such an assay system is for initial screening of candidate compounds, since the system is generically applicable to all G protein-coupled receptors without regard to the particular G protein that binds to the receptor.

Gₛ₂₀ stimulates the enzyme adenylyl cyclase, while Gᵢ and Gₒ inhibit that enzyme. As is well known the art, adenylyl cyclase catalyzes the conversion of ATP to cAMP; thus, constitutively activated GPCRs that couple the Gₛ protein are associated with increased cellular levels of cAMP. Alternatively, constitutively activated GCPRs that might couple the Gᵢ (or Gₒ) protein are associated with decreased cellular levels of cAMP. See “Indirect Mechanism of Synaptic Transmission”, Chpt. 8, from Neuron to Brain (3^{rd} Ed.), Nichols et al. eds., Sinauer Associates, Inc., 1992. Thus, assays that detect cAMP can be used to determine if a candidate compound is an inverse agonist to the receptor. A variety of approaches known in the art for measuring cAMP can be utilized. In one embodiment, anti-cAMP antibodies are used in an ELISA-based format. In another embodiment, a whole cell second messenger reporter system assay is contemplated (see PCT Publication No. WO 00/22131).

In a related aspect, cyclic AMP drives gene expression by promoting the binding of a cAMP-responsive DNA binding protein or transcription factor (CREB) which then binds to the promoter at specific sites called cAMP response elements, and drives the expression of the gene. Thus, reporter systems can be constructed which have a promoter containing multiple cAMP response elements before the reporter gene, e.g., β-galactosidase or luciferase. Further, as a constitutively activated Gₛ-linked receptor causes the accumulation of cAMP, that then activates the gene and expression of the reporter protein. The reporter protein, such as β-galactosidase or luciferase, then can be detected using standard biochemical assays (PCT Publication No. WO 00/22131).

Other G proteins, such as Gₒ and G_{q}, are associated with activation of the enzyme phospholipase C, which in turn hydrolyzes the phospholipid, PIP2, releasing two intracellular messengers: diacylglycerol (DAG) and inositol 1,4,5-triphosphate (IP3). Increased accumulation of IP3 is associated with activation of G_{q}-associated receptors and Gₒ-associated receptors (PCT Publication No. WO 00/22131). Assays that detect IP3 accumulation can be used to determine if a candidate compound is an inverse agonist to a G_{q}-associated receptor or a Gₒ-associated receptor. G_{q}-associated receptors also can be examined using an AP1 reporter assays that measures whether G_{q}-dependent phospholipase C causes activation of genes containing AP1 elements. Thus, activated G_{q}-associated receptors will demonstrate an increase in the expression of such genes, whereby inverse agonists will demonstrate a decrease in such expression.

Also provided herein is a method (also referred to herein as a “screening assay”) for identifying modulators, i.e., candidate or test compounds or agents (e.g., peptides, peptidomimetics, small molecules or other drugs) that bind to GAVE19 proteins or have a stimulatory or inhibitory effect on, for example, GAVE19 expression or GAVE19 activity.

In one embodiment, the invention provides assays for screening candidate or test compounds that bind to or modulate the activity of the membrane-bound form of a GAVE19 protein, polypeptide or biologically active portion thereof. The test compounds of the instant invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des (1997) 12:145).

Examples of methods for the synthesis of molecular libraries can be found in the art, for example in: DeWitt et al., Proc Natl Acad Sci USA (1993) 90:6909; Erb et al., Proc Natl Acad Sci USA (1994) 91:11422; Zuckermann et al., J Med Chem (1994) 37:2678; Cho et al., Science (1993) 261:1303; Carrell et al., Angew Chem Int Ed Engl (1994) 33:2059; Carell et al., Angew Chem Int Ed Engl (1994) 33:2061; and Gallop et al., J Med Chem (1994) 37:1233.

Libraries of compounds may be presented in solution (e.g., Houghten Bio/Techniques (1992) 13:412-421) or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al., Proc Natl Acad Sci USA (1992) 89:1865-1869) or phage (Scott et al., Science (1990) 249:386-390; Devlin, Science (1990) 249:404-406; Cwirla et al., Proc Natl Acad Sci USA (1990) 87:6378-6382; and Felici, J Mol Biol (1991) 222:301-310).

In a particular embodiment of the present invention, an assay is a cell-based assay in which a cell that expresses a membrane-bound form of GAVE19 protein, or a biologically active portion thereof, on the cell surface is contacted with a test compound and the ability of the test compound to bind to a GAVE19 protein is determined. The cell, for example, can be a yeast cell or a cell of mammalian origin. Determining the ability of the test compound to bind to the GAVE19 protein can be accomplished, for example, by coupling the test compound with a radioisotope or enzymatic label so that binding of the test compound to the GAVE19 protein or biologically active portion thereof can be determined by detecting the labeled compound in a complex. For example, test compounds can be labeled with ¹²⁵I, ³⁵S, ¹⁴C or ³H, either directly or indirectly and the radioisotope detected by direct counting of radioemmission or by scintillation counting. Alternatively, test compounds can be labeled enzymatically with, for example, horseradish peroxidase, alkaline phosphatase or luciferase and the enzymatic label detected by determination of conversion of an appropriate substrate to product. In a particular embodiment, the assay comprises contacting a cell that expresses a membrane-bound form of GAVE19 protein or a biologically active portion thereof, on the cell surface with a known compound that binds GAVE19 to form an assay mixture, contacting the assay mixture with a test compound and determining the ability of the test compound to interact with a GAVE19 protein, wherein determining the ability of the test compound to interact with a GAVE19 protein comprises determining the ability of the test compound to bind preferentially to GAVE19 or a biologically active portion thereof as compared to the known compound.

In another embodiment, an assay is a cell-based assay comprising contacting a cell expressing a membrane-bound form of GAVE19 protein or a biologically active portion thereof, on the cell surface with a test compound and determining the ability of the test compound to modulate (e.g., stimulate or inhibit) the activity of the GAVE19 protein or biologically active portion thereof. Determining the ability of the test compound to modulate the activity of GAVE19 or a biologically active portion thereof can be accomplished, for example, by determining the ability of the GAVE19 protein to bind to or to interact with a GAVE19 target molecule. As used herein, a “target molecule” is a molecule with which a GAVE19 protein binds or interacts in nature, for example, a molecule on the surface of a cell that expresses a GAVE19 protein, a molecule on the surface of a second cell, a molecule in the extracellular milieu, a molecule associated with the internal surface of a cell membrane or a cytoplasmic molecule. A GAVE19 target molecule can be a non-GAVE19 molecule or a GAVE19 protein or polypeptide of the instant invention. In one embodiment, a GAVE19 target molecule is a component of a signal transduction pathway that facilitates transduction of an extracellular signal (e.g., a signal generated by binding of a compound to a membrane-bound GAVE19 molecule) through the cell membrane and into the cell. The target, for example, can be a second intercellular protein that has catalytic activity or a protein that facilitates the association of downstream signaling molecules with GAVE19.

Determining the ability of the GAVE19 protein to bind to or to interact with a GAVE19 target molecule can be accomplished by one of the methods described above for determining direct binding. In a particular embodiment, determining the ability of the GAVE19 protein to bind to or to interact with a GAVE19 target molecule can be accomplished by determining the activity of the target molecule. For example, the activity of the target molecule can be determined by detecting induction of a cellular second messenger of the target (e.g., intracellular Ca²⁺, diacylglycerol, IP3 etc.), detecting catalytic/enzymatic activity of the target on an appropriate substrate, detecting the induction of a reporter gene (e.g., a GAVE19-responsive regulatory element operably linked to a nucleic acid encoding a detectable marker, e.g. luciferase) or detecting a cellular response, e.g., cellular differentiation or cell proliferation.

The present invention further extends to a cell-free assay comprising contacting a GAVE19 protein, or biologically active portion thereof, with a test compound, and determining the ability of the test compound to bind to the GAVE19 protein or biologically active portion thereof. Binding of the test compound to the GAVE19 protein can be determined either directly or indirectly as described above. In a preferred embodiment, the assay includes contacting the GAVE19 protein or biologically active portion thereof with a known compound that binds GAVE19 to form an assay mixture, contacting the assay mixture with a test compound, and determining the ability of the test compound to interact with a GAVE19 protein, wherein determining the ability of the test compound to interact with a GAVE19 protein comprises determining the ability of the test compound to preferentially bind to GAVE19 or biologically active portion thereof as compared to the known compound.

Another cell-free assay of the present invention involves contacting GAVE19 protein or biologically active portion thereof, with a test compound and determining the ability of the test compound to modulate (e.g., stimulate or inhibit) the activity of the GAVE19 protein or biologically active portion thereof. Determining the ability of the test compound to modulate the activity of GAVE19 can be accomplished, for example, by determining the ability of the GAVE19 protein to bind to a GAVE19 target molecule by one of the methods described above for determining direct binding. In an alternative embodiment, determining the ability of the test compound to modulate the activity of GAVE19 can be accomplished by determining the ability of the GAVE19 protein to further modulate a GAVE19 target molecule. For example, the catalytic/enzymatic activity of the target molecule on an appropriate substrate can be determined as described previously.

Still another cell-free assay of the present invention comprises contacting the GAVE19 protein or biologically active portion thereof, with a known compound that binds GAVE19 to form an assay mixture, contacting the assay mixture with a test compound and determining the ability of the test compound to interact with a GAVE19 protein. The step for determining the ability of the test compound to interact with a GAVE19 protein comprises determining the ability of the GAVE19 protein preferentially to bind to or to modulate the activity of a GAVE19 target molecule.

Receptors can be activated by non-ligand molecules that necessarily do not inhibit ligand binding but cause structural changes in the receptor to enable G protein binding or, perhaps receptor aggregation, dimerization or clustering that can cause activation. For example, antibodies can be raised to the various portions of GAVE19 that are exposed at the cell surface. Those antibodies activate a cell via the G protein cascade as determined by standard assays, such as monitoring cAMP levels or intracellular Ca⁺² levels. Because molecular mapping, and particularly epitope mapping, is involved, monoclonal antibodies may be preferred. The monoclonal antibodies can be raised both to intact receptor expressed at the cell surface and peptides known to form at the cell surface. The method of Geysen et al., U.S. Pat. No. 5,998,577, can be practiced to obtain a plurality of relevant peptides. Antibodies found to activate GAVE19 may be modified to minimize activities extraneous to GAVE19 activation, such as complement fixation. Thus, the antibody molecules can be truncated or mutated to minimize or to remove activities outside of GAVE19 activation. For example, for certain antibodies, only the antigen-binding portion is needed. Thus, the F_{c} portion of the antibody can be removed.

Cells expressing GAVE19 are exposed to antibody to activate GAVE19. Activated cells then are exposed to various molecules in order to identify which molecules modulate receptor activity, and result in higher activation levels or lower activation levels. Molecules that achieve those goals then can be tested on cells expressing GAVE19 without antibody to observe the effect on non-activated cells. The target molecules then can be tested and modified as candidate drugs for the treatment of disorders associated with altered GPCR metabolism using known techniques.

The cell-free assays of the instant invention are amenable for use of both the soluble form and the membrane-bound form of GAVE19. In the case of cell-free assays comprising the membrane-bound form of GAVE19, it may be desirable to utilize a solubilizing agent such that the membrane-bound form of GAVE19 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON X-100, TRITON X-114, THESIT, isotridecylpoly(ethylene glycol ether)ₙ, 3-[(3-cholamidopropyl)dimethylammino]-1-propane sulfonate (CHAPS), 3-[(3-cholamidopropyl)dimethylammino]-2-hydroxy-1-propane sulfonate (CHAPSO) or N-dodecyl=N,N-dimethyl-3-ammonio-1-propane sulfonate.

In more than one embodiment of the above assay methods of the instant invention, it may be desirable to immobilize either GAVE19 or a target molecule thereof to facilitate separation of complexed from uncomplexed forms of one or both of the proteins, as well as to accommodate automation of the assay. Binding of a test compound to GAVE19 or interaction of GAVE19 with a target molecule in the presence and absence of a candidate compound, can be accomplished in any vessel suitable for containing the reactants. Examples of such vessels include microtitre plates, test tubes and micro-centrifuge tubes. In one embodiment, a fusion protein can be provided that adds a domain that allows one or both of the proteins to be bound to a matrix. For example, glutathione-S-transferase/GAVE19 fusion proteins or glutathione-S-transferase/target fusion proteins can be adsorbed onto glutathione SEPHAROSE beads (Sigma Chemical, St. Louis, Mo.). Alternatively, glutathione-derivatized microtitre plates are then combined with the test compound. Subsequently, either the non-adsorbed target protein or GAVE19 protein and the mixture are incubated under conditions conducive to complex formation (e.g., at physiological conditions for salt and pH). Following incubation, the beads or microtitre plate wells are washed to remove any unbound components, and the presence of complex formation is measured either directly or indirectly. Alternatively, the complexes can be dissociated from the matrix and the level of GAVE19 binding or activity determined using standard techniques.

Other techniques for immobilizing proteins on matrices can also be used in the screening assays of the invention. For example, either GAVE19 or a target molecule thereof can be immobilized utilizing conjugation of biotin and streptavidin. Biotinylated GAVE19 or target molecules can be prepared from biotin-NHS (N-hydroxy-succinimide) using techniques well known in the art (e.g., biotinylation kit, Pierce Chemicals, Rockford, Ill.) and immobilized in the wells of streptavidin-coated 96-well plates (Pierce Chemicals). Alternatively, antibodies that are reactive with GAVE19 or a target molecule, but do not interfere with binding of the GAVE19 protein to the target molecule, can be derivatized to the wells of the plate. Upon incubation, unbound target or GAVE19 can be trapped in the wells by antibody conjugation. Methods for detecting such complexes, in addition to those described above for the GST-immobilized complexes, include immunodetection of complexes using antibodies reactive with GAVE19 or target molecule, as well as enzyme-linked assays that rely on detecting an enzymatic activity associated with the GAVE19 or target molecule.

In another embodiment, modulators of GAVE19 expression are identified in a method wherein a cell is contacted with a candidate compound, and the expression of GAVE19 mRNA or protein in the cell is determined. The level of expression of GAVE19 mRNA or protein in the presence of the candidate compound is compared to the level of expression of GAVE19 mRNA or protein in the absence of the candidate compound. The candidate compound then can be identified as a modulator of GAVE19 expression based on that comparison. For example, when expression of GAVE19 mRNA or protein is greater (statistically significantly greater) in the presence of the candidate compound than in the absence thereof, the candidate compound is identified as a stimulator or agonist of GAVE19 mRNA or protein expression. Alternatively, when expression of GAVE19 mRNA or protein is less (statistically significantly less) in the presence of the candidate compound than in the absence thereof, the candidate compound is identified as an inhibitor or antagonist of GAVE19 mRNA or protein expression. If GAVE19 activity is reduced in the presence of ligand or agonist, or in a constitutive GAVE19, below baseline, the candidate compound is identified as an inverse agonist. The level of GAVE19 mRNA or protein expression in the cells can be determined by methods described herein for detecting GAVE19 mRNA or protein.

In yet another aspect of the invention, the GAVE19 proteins can be used as “bait proteins” in a two-hybrid assay or three-hybrid assay (see, e.g., U.S. Pat. No. 5,283,317; Zervos et al., Cell (1993) 72:223-232; Madura et al., J Biol Chem (1993) 268:12046-12054; Bartel et al., Bio/Techniques (1993) 14:920-924; Iwabuchi et al., Oncogene (1993) 8:1693-1696; and PCT Publication No. WO 94/10300), to identify other proteins that bind to or interact with GAVE19 (“GAVE19-binding proteins” or “GAVE19-bp”), and modulate GAVE19 activity. Such GAVE19-binding proteins are also likely to be involved in the propagation of signals by the GAVE19 proteins such as, for example, upstream or downstream elements of the GAVE19 pathway.

Since the present invention enables the production of large quantities of pure GAVE19, physical characterization of the conformation of areas of likely function can be ascertained for rational drug design. For example, the IC3 region of the molecule and EC domains are regions of particular interest. Once the shape and ionic configuration of a region is discerned, candidate drugs that should interact with those regions can be configured and then tested in intact cells, animals and patients. Methods that would enable deriving such 3-D structure information include X-ray crystallography, NMR spectroscopy, molecular modeling and so on. The 3-D structure also can lead to identification of analogous conformational sites in other known proteins where known drugs that act at site exist. Those drugs, or derivatives thereof, may find use in treating inflammatory diseases or disorders such as rheumatoid arthritis or COPD, to name only a few.

The invention further pertains to novel agents identified by the above-described screening assays and uses thereof for treatments as described herein.

### Assays of the Present Invention

A. Detection Assays

Portions or fragments of the DNA sequences of the present invention can be used in numerous ways as polynucleotide reagents. For example, the sequences can be used to: (i) map the respective genes on a chromosome and, thus, locate gene regions associated with genetic disease; (ii) identify an individual from a minute biological sample (tissue typing); and (iii) aid in forensic identification of a biological sample. The applications are described in the subsections below.

1. Chromosome Mapping

Once the sequence (or a portion of the sequence) of a gene has been isolated, the sequence can be used to map the location of the GAVE19 gene on a chromosome. Accordingly, GAVE19 nucleic acid molecules described herein or fragments thereof can been used to map the location of GAVE19 in a genome. The mapping of the location of the GAVE19 sequence in a genome is an important step in correlating the sequences with genes associated with disease.

Briefly, GAVE19 genes can be mapped in a genome by preparing PCR primers (preferably 15-25 bp in length) from the GAVE19 sequences. The primers are used for PCR screening of somatic cell hybrids containing individual murine chromosomes. Only those hybrids containing the murine gene corresponding to the GAVE19 sequences yield an amplified fragment.

A particular method includes, but certainly is not limited to denaturing murine chromosomes and then contacting them with a detectably labeled GAVE19 DNA molecule under stringent hybridization conditions. Hybridization and subsequent detection of the detectably labeled GAVE19 DNA molecule will reveal the location of GAVE 19 in the murine chromosome. Such an in situ hybridization technique is described in Fan et al., Proc Natl Acad Sci USA (1990) 87:6223-27, which involves pre-screening with labeled flow-sorted chromosomes and pre-selection by hybridization to chromosome-specific cDNA libraries. The in situ hybridization (FISH) of a DNA sequence to a metaphase chromosomal spread can also be used to provide a precise chromosomal location in one step. Chromosome spreads can be made using cells in which division has been blocked in metaphase by a chemical, e.g., colcemid, that disrupts the mitotic spindle. The chromosomes can be treated briefly with trypsin and then stained with Giemsa. A pattern of light and dark bands develops on each chromosome so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases and more preferably, 2,000 bases will suffice to get good results in a reasonable amount of time. For a review of the technique, see Verma et al. (Human Chromosomes: A Manual of Basic Techniques (Pergamon Press, New York, 1988)). Chromosomal mapping can be inferred in silico, and employing statistical considerations, such as lod scores or mere proximity.

Reagents for chromosome mapping can be used individually to locate a single site on a chromosome. Furthermore, panels of reagents can be used for marking multiple sites and/or multiple chromosomes. Reagents corresponding to flanking regions of the GAVE19 gene actually are preferred for mapping purposes. Coding sequences are more likely to be conserved within gene families, thus increasing the chance of cross hybridization during chromosomal mapping.

Once a sequence has been mapped to a precise chromosomal location, the physical position of the sequence on the chromosome can be correlated with genetic map data. The relationship between genes and disease, mapped to the same chromosomal region, can then be identified through linkage analysis (co-inheritance of physically adjacent genes), described in, e.g., Egeland et al., Nature (1987) 325:783-787.

Moreover, differences in the DNA sequences between animals affected and unaffected with a disease associated with GAVE19 can be determined. If a mutation is observed in some or all of the affected animals, but not in any unaffected animals, then the mutation is likely to be the causative agent of the particular disease. Comparison of affected and unaffected animals generally involves first looking for structural alterations in the chromosomes such as deletions or translocations that are visible from chromosome spreads or detectable using PCR based on that DNA sequence. Ultimately, complete sequencing of genes from several animals can be performed to confirm the presence of a mutation and to distinguish mutations from polymorphisms.

1. Diagnostic Assays

An exemplary method for detecting the presence or absence of GAVE19 in a biological sample involves obtaining a biological sample from a test animal and contacting the biological sample with a compound or an agent capable of detecting GAVE19 protein or nucleic acid (e.g., mRNA or genomic DNA) that encodes GAVE19 protein such that the presence of GAVE19 is detected in the biological sample. A preferred agent for detecting GAVE19 mRNA or genomic DNA is a labeled nucleic acid probe capable of hybridizing to GAVE19 mRNA or genomic DNA. The nucleic acid probe can be, for example, a full-length GAVE19 nucleic acid, such as the nucleic acid of SEQ ID NO:1 or a portion thereof, such as an oligonucleotide of at least 15, 30, 50, 100, 250 or 500 or more nucleotides in length and sufficient to specifically hybridize under stringent conditions to GAVE19 mRNA or genomic DNA. Other suitable probes for use in the diagnostic assays of the invention are described herein.

A particular agent for detecting GAVE19 protein is an antibody capable of binding to GAVE19 protein, preferably an antibody with a detectable label. Antibodies can be polyclonal, chimeric, or more preferably, monoclonal. An intact antibody or a fragment thereof (e.g., F_{ab} or F_{(ab′)2}) can be used. The term “biological sample” is intended to include tissues, cells and biological fluids isolated from a subject, as well as tissues, cells and fluids present within a subject. That is, the detection method of the invention can be used to detect GAVE19 mRNA, protein or genomic DNA in a biological sample in vitro as well as in vivo. For example, in vitro techniques for detection of GAVE19 mRNA include Northern hybridization and in situ hybridization. In vitro techniques for detection of GAVE19 protein include ELISA, Western blot, immunoprecipitation and immunofluorescence. In vitro techniques for detection of GAVE19 genomic DNA include Southern hybridization.

Furthermore, in vivo techniques for detection of GAVE19 protein include introducing into an animal a labeled anti-GAVE19 antibody. For example, the antibody can be labeled with a radioactive marker, the presence and location of which in an animal can be detected by standard imaging techniques.

In an embodiment, the biological sample contains protein molecules from the test animal. Alternatively, the biological sample can contain mRNA molecules from the test animal or genomic DNA molecules from the test animal. A particular biological sample having applications herein is a peripheral blood leukocyte sample isolated by conventional means from an animal.

In another embodiment, the methods further involve obtaining a biological sample from a control animal, contacting the control sample with a compound or agent capable of detecting GAVE19 protein, mRNA or genomic DNA, such that the presence and amount of GAVE19 protein, mRNA or genomic DNA is detected in the biological sample, and then comparing the presence and amount of GAVE19 protein, mRNA or genomic DNA in the control sample with the presence and amount of GAVE19 protein, mRNA or genomic DNA in a test sample to determine whether the compound modulates the expression or activity of GAVE 19.

### High Throughput Assays of Chemical Libraries

Any of the assays for compounds capable of modulating the activity of GAVE19 are amenable to high throughput screening. High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### Kits

The invention also encompasses kits for detecting the presence of GAVE19 in a biological sample (a test sample). Such kits can be used to determine whether a particular compound modulates the expression or activity of GAVE19. For example, the kit can comprise a labeled compound or agent capable of detecting GAVE19 protein or mRNA in a biological sample and means for determining the amount of GAVE19 in the sample (e.g., an anti-GAVE19 antibody or an oligonucleotide probe that binds to DNA encoding GAVE19, e.g., SEQ ID NO:1).

For antibody-based kits, the kit can comprise, for example: (1) a first antibody (e.g., attached to a solid support) that binds to GAVE19 protein; and, optionally, (2) a second, different antibody that binds to GAVE19 protein or to the first antibody and is conjugated to a detectable agent. If the second antibody is not present, then either the first antibody can be detectably labeled, or alternatively, another molecule that binds the first antibody can be detectably labeled. In any event, a labeled binding moiety is included to serve as the detectable reporter molecule, as known in the art.

For oligonucleotide-based kits, a kit of the present invention can comprise, for example: (1) an oligonucleotide, e.g., a detectably-labeled oligonucleotide, that hybridizes to a GAVE19 nucleic acid sequence or (2) a pair of primers useful for amplifying a GAVE19 nucleic acid molecule.

The kit also can comprise, e.g., a buffering agent, a preservative or a protein stabilizing agent. The kit also can comprise components necessary for detecting the detectable agent (e.g., an enzyme or a substrate). Furthermore, the kit may also contain a control sample or series of control samples that can be assayed and compared to the test sample. Each component of the kit is usually enclosed within an individual container, and all of the various containers are within a single package.

2. Pharmacogenomics

As explained herein, GAVE19 expression is modulated in cells associated with activated or inflammatory states. Disorders associated with inflammation include, anaphylactic states, colitis, Crohn's Disease, edematous states, contact hypersensitivity, allergy, other forms of arthritis, meningitis and other conditions wherein the immune system reacts to an insult by vascular dilation, heat, collecting cells, fluids and the like at a site resulting in swelling and the like. Thus, agents or modulators that have a stimulatory or inhibitory effect on GAVE19 activity (e.g., GAVE19 gene expression) as identified by a screening assay described herein can be administered to individuals to treat (prophylactically or therapeutically) disorders (e.g., inflammation associated with asthma, chronic obstructive pulmonary disease and rheumatoid arthritis). In conjunction with such treatment, the pharmacogenomics (i.e., the study of the relationship between the genotype of an individual and the response of the individual to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the genotype of the individual. Such pharmacogenomics further can be used to determine appropriate dosages and therapeutic regimens.

Pharmacogenomics deals with clinically significant hereditary variations in the response to drugs due to altered drug disposition and abnormal action in affected persons. See, e.g., Linder, Clin Chem (1997) 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” The pharmacogenetic conditions can occur either as rare defects or as polymorphisms. For example, glucose-6-phosphate dehydrogenase deficiency (G6PD) is a common inherited enzymopathy in that the main clinical complication is hemolysis after ingestion of oxidant drugs (anti-malarials, sulfonamides, analgesics or nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes, CYP2D6 and CYP2C19) has provided an explanation as to why some patients do not obtain the expected drug effects or show exaggerated drug response and serious toxicity after taking the standard and safe dose of a drug. The polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, all which lead to the absence of functional CYP2D6. Poor metabolizers of CYP2D6 and CYP2C19 quite frequently experience exaggerated drug response and side effects when standard doses are received. If a metabolite is the active therapeutic moiety, a PM will show no therapeutic response, as demonstrated for the analgesic effect of codeine mediated by the CYP2D6-formed metabolite, morphine. The other extreme is the so-called ultra-rapid metabolizers that do not respond to standard doses. Recently, the molecular basis of ultra-rapid metabolism has been identified to be due to CYP2D6 gene amplification.

Activity of a homolog of GAVE19 protein, or expression of a DNA molecule that is a homolog of GAVE19 nucleic acid in an individual can be determined to select thereby appropriate agent(s) for therapeutic or prophylactic treatment of the individual. In addition, pharmacogenetic studies can be used to apply genotyping of polymorphic alleles encoding drug-metabolizing enzymes to the identification of the drug responsiveness phenotype of an individual. That knowledge, when applied to dosing or drug selection, can avoid adverse reactions or therapeutic failure and thus enhance therapeutic or prophylactic efficiency when treating a subject with a GAVE19 modulator, such as a modulator identified by one of the exemplary screening assays described herein.

D. Methods of Treatment

As explained above, the present invention extends to assays for identifying drugs or agents that modulate the expression of GAVE19 in mice. Due to the expression profile of GAVE19, i.e. is highly expressed in normal spleen, has an expression level that is increased two fold relative to normal spleen in collagen induced arthritis (CIA) RA mouse spleen, and has an expression level elevated over five-fold in CIA RA mouse lung compared with normal lung, such drugs or agents may readily have applications in treating diseases or disorders such as inflammatory disorders (e.g. asthma), chronic obstructive pulmonary disease and rheumatoid arthritis, to name only a few.

1. Prophylactic Methods

In one aspect, the invention provides a method for preventing in a subject, a disease or condition such as those described above, by administering to the subject an agent that modulates GAVE19 expression or at least one GAVE19 activity. Subjects that may benefit from such treatment can be identified by, for example, any or a combination of diagnostic or prognostic assays well known to those of ordinary skill in the art. Administration of a prophylactic agent can occur prior to the manifestation of symptoms for such a disease or disorder in order to prevent the disease or disorder, or alternatively to delay its progression.

2. Therapeutic Methods

An agent that can used for treating inflammatory diseases, i.e., that is found to modulate GAVE19 protein activity in mice, can be an agent as described herein, such as a nucleic acid or a protein, a naturally-occurring cognate ligand of a GAVE19 protein, a peptide, a GAVE19 peptidomimetic or other small molecule. In one embodiment, the agent stimulates one or more of the biological activities of GAVE 19 protein. Examples of such stimulatory agents include active GAVE19 protein and a nucleic acid molecule encoding GAVE19 that has been introduced into the cell. In another embodiment, the agent inhibits one or more of the biological activities of GAVE19 protein. Examples of such inhibitory agents include antisense GAVE19 nucleic acid molecules and anti-GAVE19 antibodies. The modulatory methods can be performed in vitro (e.g., by culturing the cell with the agent) or, alternatively, in vivo (e.g., by administering the agent to a subject). As such, the instant invention provides methods of treating an individual afflicted with a disease or disorder as described above that comprises administering to the individual an agent (e.g., an agent identified by a screening assay described herein) or combination of agents that modulates (e.g., upregulates or downregulates) GAVE19 expression or activity in mice. In another embodiment, the method involves administering a GAVE19 protein or nucleic acid molecule as therapy.

The present invention may be better understood by reference to the following non-limiting Example, which is provided as exemplary of the invention. The following Example is presented in order to more fully illustrate the preferred embodiments of the invention. It should in no way be construed, however, as limiting the broad scope of the invention.

### EXAMPLE

The large number of G-protein coupled receptors are the target of ˜50% of the current therapeutic drugs on the market. The GPCRs are activated by a wide variety of ligands, including peptide, neurotransmitters, hormones, growth factors, amines, lipids, fatty acids, odorant molecules and ligts. The perturbation of GPCR function results in many pathological conditions. GAVE19, the ortholog of human GAVE18, is highly expressed in normal spleen, has an expression level that is increased two fold relative to normal spleen in collagen induced arthritis (CIA) RA mouse spleen, and has an expression level elevated over five-fold in CIA RA mouse lung compared with normal lung. Thus, GAVE19 provides a new drug target drugs or agents for treating diseases or disorders such as inflammatory disorders (e.g. asthma), chronic obstructive pulmonary disease and rheumatoid arthritis, to name only a few.

Identification and cloning of GAVE19 also provides the opportunity to find the endogenous ligand through a process “de-orphaning”. Natural ligand or surrogate ligand identified through de-orphaning provides a tool for screening some molecules which activate or block the receptor signal transduction, and therefore change the cellular physiology and cell function.

Materials and Methods

Identification and cloning of GAVE19. Using human GAVE18 DNA sequence to quire mouse genomic DNA database did not reveal the mouse homolog DNA sequence. Human GAVE18 DNA containing coding region was then used as the probe to screen Research Genetics mouse genomic BAC libraries. Multiple DNA primers designed according human GAVE18 sequence were used to sequence the positive mouse BAC. Only the primers, 5′ GGC TTC CCC CAA AGA CAA AG 3′ (SEQ ID NO:3) gave the mouse GAVE19 DNA sequence data. Multiple mouse DNA sequencing primers were then designed for primer walking to sequence the mouse GAVE19 coding region.

Mouse disease models and RNA isolation. Mouse total RNAs isolated from different tissues and organs are performed with Trizol reagent from GIBCO BLR according manufactory instruction. The total RNAs were converted to cDNAs by using Multiscribe RT-PCR kit from ABI.

TaqMan analysis. TaqMan reactions are performed in duplicate with mouse tissue cDNA and GAPDH gene is used as an internal control. TaqMan results are then calculated as relative expression. The relative expression equals (2´(O-ddCt))*1000, where ddCT equals (GAVE19 mean Cts-GAPDH mean Cts)−(GAVE19 mean NTC Cts-GAPDH mean NTC Cts). TaqMan probe was custom synthesized by Operon Technologies. TaqMan primer sequence 1: 5′ CTGTTCTTGCTGGTGAAAATGAA 3′ (SEQ ID NO:4) and sequence 2: 5′ CCATGAACCACCACGAGGTT 3′ (SEQ ID NO:5). TaqMan probe sequence: 5′ (Fam)-TCACGTTCAGTGACCACCATGGCTG-(Tet) 3 ′ (SEQ ID NO:6). TaqMan reaction was performed in a 96-well plate MicroAmp optical tube (PE).

### Description of the Results

TaqMan expression profile showed gradualy increased expression levels of GAVE19 in the joints of CIA RA model from normal controled sample to RA grade 4. The higher RA grade in the joints correlates higher GAVE19 expression. GAVE19 also showed dramatically increased expression levels in CIA RA lung and spleen to compare with normal controled lung and spleen. In mouse EAE (Experimental allergic encephalomyelitis) model of multiple sclerosis, GAVE19 showed increased expression level in the brain during the EAE peak stage to compare with the expression level in the brain during preclinic stage.

Table of TaqMan Expression Profile of GAVE19 in Mouse Tissues

| Tissue Type | CT | Mean | GAPDH CT | GAPDH Mean | | | Expression |
|---|---|---|---|---|---|---|---|
| EAE Control (Unlmm.) Brain | 34.78 | 34.58 | 19.04 | 19.35 | 15.24 | 15.24 | 0.03 |
| | 34.38 | | 19.65 | | | | |
| EAE Control (Unlmm.) Heart | 35.63 | 35.86 | 22.12 | 22.07 | 13.80 | 13.80 | 0.07 |
| | 36.09 | | 22.01 | | | | |
| EAE Control (Unimm.) Liver | 36.81 | 36.80 | 20.63 | 20.93 | 15.87 | 15.87 | 0.02 |
| | 36.78 | | 21.22 | | | | |
| EAE Vehicle Brain | 34.21 | 34.12 | 19.12 | 19.09 | 15.04 | 15.04 | 0.03 |
| | 34.03 | | 19.05 | | | | |
| EAE Vehicle Heart | 33.01 | 32.64 | 19.13 | 18.66 | 13.98 | 13.98 | 0.06 |
| | 32.26 | | 18.18 | | | | |
| EAE Vehicle Liver | 35.37 | 35.27 | 20.86 | 20.70 | 14.57 | 14.57 | 0.04 |
| | 35.16 | | 20.54 | | | | |
| EAE Preclinical, d7 Brain | 35.06 | 35.10 | 19.94 | 19.99 | 15.12 | 15.12 | 0.03 |
| | 35.14 | | 20.03 | | | | |
| EAE Preclinical, d7 Heart | 34.33 | 34.32 | 19.05 | 18.99 | 15.33 | 15.33 | 0.02 |
| | 34.3 | | 18.93 | | | | |
| EAE Preclinical, d7 Liver | 34.15 | 34.24 | 20.23 | 20.33 | 13.91 | 13.91 | 0.06 |
| | 34.33 | | 20.43 | | | | |
| EAE Peak Brain | 30.75 | 30.73 | 19.96 | 19.93 | 10.80 | 10.80 | 0.56 |
| | 30.7 | | 19.9 | | | | |
| EAE Peak Heart | 32.82 | 32.75 | 18.84 | 19.09 | 13.67 | 13.67 | 0.08 |
| | 32.68 | | 19.33 | | | | |
| EAF Peak Liver | 32.61 | 32.41 | 19.93 | 20.48 | 11.93 | 11.93 | 0.26 |
| | 32.2 | | 21.03 | | | | |
| EAE Sustained Remission | 33.6 | 33.52 | 19.61 | 19.80 | 13.73 | 13.73 | 0.07 |
| | 33.44 | | 19.98 | | | | |
| EAE Relapse Brain | 34.32 | 34.42 | 20.89 | 20.71 | 13.71 | 13.71 | 0.07 |
| | 34.51 | | 20.52 | | | | |
| EAE (Unimm.) Lung | 32.04 | 32.11 | 22 | 22.46 | 9.66 | 9.66 | 1.24 |

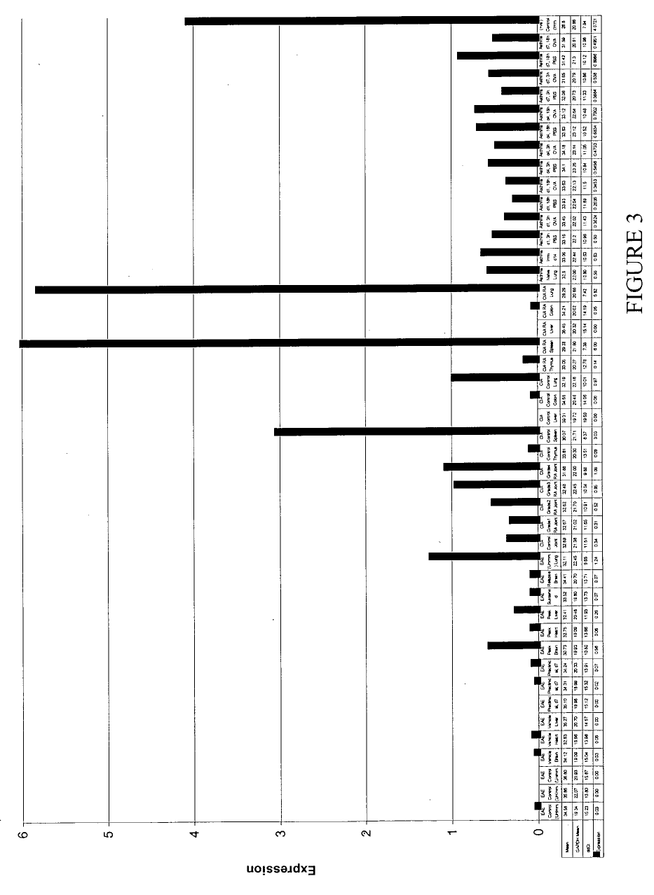

Expression profile data is also graphically shown in FIG. 3. All of these data point that GAVE 19 plays important roles in inflammation related diseases.

### Discussion

A novel mouse G-protein coupled receptor, GAVE19, which is the ortholog of human GAVE18 has been identified and cloned. Full-length coding region of the DNA and its deduced amino acid sequence have been characterized. Tissue distribution of the receptor has been analyszed by RT-PCR (TaqMan) analyses in normal controled mice and collegen induced arthritis (CIA) mice. Expression profiles of GAVE19 show the similarities to GAVE18 (the human ortholog) and clearly indicate its roles in inflammation diseases such as rheumatoid arthritis. Hence, using various assays described herein, GAVE19 provides a new drug target for inflammation diseases, such as asthma, RA, COPD, etc. Moreover, compounds and agents found with assays of the present invention to modulate the expression and/or activity of GAVE19 in mice may readily applications in treating such diseases.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. An isolated nucleic acid molecule comprising the DNA sequence of FIG. 1 (SEQ ID NO:1).

2. An isolated nucleic acid molecule hybridizable to said isolated nucleic acid molecule of claim 1, or a hybridization probe that is complementary to said isolated nucleic acid molecule of claim 1, under stringent hybridization conditions.

3. The isolated nucleic acid molecule of either of claims 1 or 2 which encodes a polypeptide having an amino acid sequence of FIG. 2 (SEQ ID NO:2).

4. The isolated nucleic acid molecule of claim 2, which encodes a polypeptide having an amino acid sequence that is at least 30% identical to said amino acid sequence of SEQ ID NO:2.

5. The isolated nucleic acid molecule of either of claims 1 or 2, which is detectably labeled.

6. The detectably labeled isolated nucleic acid molecule of claim 5, wherein said detectable label comprises an enzyme, a radioactive isotope, or a chemical which fluoresces.

7. A purified polypeptide comprising the amino acid sequence of FIG. 2 (SEQ ID NO:2).

8. An isolated nucleic acid molecule which encodes said purified polypeptide of claim 7.

9. The purified polypeptide of claim 7 which is detectably labeled.

10. The purified polypeptide of claim 9, wherein said detectable label comprises an enzyme, a radioactive isotope, or a chemical which fluoresces.

11. An antibody having said purified polypeptide of claim 7 as an immunogen.

12. The antibody of claim 11, wherein said antibody is selected from the group consisting of a monoclonal antibody, a polyclonal antibody, or a chimeric antibody.

13. The antibody of claim 11, which is detectably labeled.

14. The antibody of claim 13, wherein said detectable label comprises an enzyme, a radioactive isotope, or a chemical which fluoresces.

15. An expression vector comprising said isolated nucleic acid molecule of claim 1 operatively associated with an expression control element.

16. An expression vector comprising said isolated nucleic acid molecule of claim 2, operatively associated with an expression control element.

17. The expression vector of either of claims 15 or 16, wherein said expression control element is selected from the group consisting of a constitutive regulatory sequence, a cell-specific regulatory sequence, and an inducible regulatory sequence.

18. The expression vector of claim 17, wherein said expression control element is a promoter.

19. The expression vector of claim 18, wherein said promoter comprises an immediate early promoter of hCMV, an early promoter of SV40, an early promoter of adenovirus, an early promoter of vaccinia, an early promoter of polyoma, a late promoter of SV40, a late promoter of adenovirus, a late promoter of vaccinia, a late promoter of polyoma, a lac system, a trp system, a TAC system, a TRC system, a major operator and promoter region of phage lambda, a control region of fd coat protein, 3-phosphoglycerate kinase promoter, acid phosphatase promoter, or a promoter of yeast α mating factor.

20. A host cell transformed or transfected with the expression vector of either of claims 15 or 16.

21. The host cell of claim 20, wherein said host cell comprises a prokaryotic cell or eukaryotic cell.

22. The host cell of claim 21, wherein said host comprises E. coli, Pseudonomas, Bacillus, Strepomyces, yeast, CHO, R1.1, B-W, L-M, COS1, COS7, BSC1, BSC40, BMT10 or Sf9 cells.

23. A method for producing the purified polypeptide of claim 7, comprising the steps of:
a) culturing a host cell of claim 20 under conditions that provide for expression of said isolated polypeptide; and
b) recovering said isolated polypeptide from said host, said culture, or a combination thereof.

24. A method for identifying an agonist of GAVE19 comprising: contacting a potential agonist with a cell expressing GAVE19 and determining whether in the presence of said potential agonist the signaling activity of GAVE19 is increased relative to the activity of GAVE19 in the absence of said potential agonist.

25. A method for identifying an inverse agonist of GAVE19 comprising: contacting a potential inverse agonist with a cell expressing GAVE19 and determining whether in the presence of said potential inverse agonist, the activity of GAVE19 is decreased relative to the activity of GAVE19 in the absence of said potential inverse agonist, and is decreased in the presence of an endogenous ligand or agonist.

26. A method for identifying an antagonist of GAVE19 comprising: contacting a potential antagonist with a cell expressing GAVE19 and determining whether in the presence of said potential antagonist the signaling activity of GAVE19 is decreased relative to the activity of GAVE19 in the presence of an endogenous ligand or agonist.

27. A therapeutic composition comprising an agonist, an antagonist, or an inverse agonist of GAVE19 capable of modulating GAVE19 signaling activity or transduction.

28. A method for treating a disease comprising administering to a patient in need of treatment a therapeutic composition comprising an agonist, an antagonist or an inverse agonist of GAVE19 capable of modulating GAVE19 signaling activity or transduction.


SEQUENCE LISTING (7 sequences)

SEQ ID NO: 1 - DNA, 810 bp, Mus musculus
atggatggatataatacctctgagaattcctcttgtgaccctatactggcacaccacttaacatcgatttacttcatagtgctcattggaggactggtaggtctcatctccatcctgttcttgctggtgaaaatgaactcacgttcagtgaccaccatggctgtcatcaacctcgtggtggttcatggggtcttcctactgacggtgcctttccgcttggcatacctcatcaaagggacttggacgtttggattacccttctgcaaatttgtgagtgccatgttacatatccacatgtacctcacgttcctcttctacgtggtgatactagtcatcagatacctcatcttcttcaagcgtagagacaaagtagaattctatagaaaattgcatgcagttgctgcaagttctgccatgtggcttctggtgattgttattgttgtgcccttggtggtttctcagtatggaaatagcgaagaatacaatgagcaacagtgctttagattccataaagaacttggccatgattctgtgcgagttatcaactatatgatagtcattgttgtcatagctgttgcgttgattctcttgggtttccaggtcttcatcacattgtccatggtgcggaagtttcgccactccttactatcccaccaggagttctgggcacaactgaaaaatcttttctttataggtatcattattatttgttttcttccctaccagttcttcaggatttattacttgtatgttgtggcacattccaagagctgtaaaaacaaagttgcattttacaatgaaatcggttga

SEQ ID NO: 2 - protein, 269 aa, Mus musculus
MDGYNTSENSSCDPILAHHLTSIYFIVLIGGLVGLISILFLLVKMNSRSVTTMAVINLVVVHGVFLLTVPFRLAYLIKGTWTFGLPFCKFVSAMLHIHMYLTFLFYVVILVIRYLIFFKRRDKVEFYRKLHAVAASSAMWLLVIVIVVPLVVSQYGNSEEYNEQQCFRFHKELGHDSVRVINYMIVIVVIAVALILLGFQVFITLSMVRKFRHSLLSHQEFWAQLKNLFFIGIIIICFLPYQFFRIYYLYVVAHSKSCKNKVAFYNEIG

SEQ ID NO: 3 - DNA, 20 bp, Artificial
  Primer
ggcttcccccaaagacaaag

SEQ ID NO: 4 - DNA, 23 bp, Artificial
  Primer
ctgttcttgctggtgaaaatgaa

SEQ ID NO: 5 - DNA, 20 bp, Artificial
  Primer
ccatgaaccaccacgaggtt

SEQ ID NO: 6 - DNA, 25 bp, Artificial
  Probe
tcacgttcagtgaccaccatggctg

SEQ ID NO: 7 - protein, 305 aa, Homo sapiens
MPGHNTSRNSSCDPIVTPHLISLYFIVLIGGLVGVISILFLLVKMNTRSVTTMAVINLVVVHSVFLLTVPFRLTYLIKKTWMFGLPFCKFVSAMLHIHMYLTFLFYVVILVTRYLIFFKCKDKVEFYRKLHAVAASAGMWTLVIVIVVPLVVSRYGIHEEYNEEHCFKFHKELAYTYVKIINYMIVIFVIAVAVILLVFQVFIIMLMVQKLRHSLLSHQEFWAQLKNLFFIGVILVCFLPYQFFRIYYLNVVTHSNACNSKVAFYNEIFLSVTAISCYDLLLFVFGGSHWFKQKIIGLWNCVLCR